(19) United States
(12) Patent Application Publication
(10) Pub. No.: US 2020/0363425 A1
(43) Pub. Date: Nov. 19, 2020

(54) ACID-MEDIATED ASSAY FOR ANALYZING LIGAND-DRUG CONJUGATES

(71) Applicant: SEATTLE GENETICS, INC., Bothell, WA (US)

(72) Inventors: Stephen C. Alley, Bothell, WA (US); Russell Sanderson, Bothell, WA (US)

(21) Appl. No.: 16/765,059

(22) PCT Filed: Nov. 20, 2018
(86) PCT No.: PCT/US2018/062100

§ 371 Date: May 18, 2020

Related U.S. Application Data
(60) Provisional application No. 62/590,169, filed on Nov. 22, 2017.

(51) Int. Cl. G01N 33/68; C07K 16/28

(52) U.S. Cl. CPC G01N 33/6848; C07K 2317/524; C07K 2317/24; C07K 16/2875

(57) ABSTRACT

Methods of analyzing a ligand-drug conjugate using acid-mediated cleavage and for implementing the methods are provided herein. Further provided include various application of the methods for analysis and development of a ligand-drug conjugate.

### CROSS REFERENCE TO RELATED APPLICATIONS

This application claims the benefit of U.S. Patent Application 62/590,169 filed Nov. 22, 2017, the disclosure of which is hereby incorporated in its entirety for all purposes

### BACKGROUND

Ligand-drug conjugates (LDCs) are the focus of increasing interest for targeted therapy. LDCs are comprised of a cytotoxic agent, typically a small molecule drug with a high systemic toxicity, and a highly selective ligand for a tissue or cell-specific antigen (e.g. an antibody in the case of antibody-drug conjugates (ADCs)), linked together through a linker that is relatively stable in circulation, but releases the cytotoxic agent in the targeted environment. Antibody-drug conjugates (ADCs) hold great promise, especially in oncology, as the next generation of targeted therapies. Leveraging the immunologic specificity of antibodies to deliver highly potent cytotoxic agents to diseased tissue both improves antitumor activity and limits off target toxicities. This approach has now been used successfully in two FDA-approved ADCs, namely brentuximab vedotin and ado-trastuzumab emtansine (Verma et al., 2012, Younes et al., 2010), and is the focus of numerous preclinical studies and clinical trials.

Intense research effort has been directed towards improving pharmacokinetic profiles, toxicity and chemical stability of LDCs. Most LDCs are heterogeneous mixtures of variably drug-loaded ligands, meaning a variable number of drug or drug-linker molecules can be linked to one ligand. Once an LDC is placed in a biological environment, biotransformations, such as loss of drug or drug-linker can occur, resulting in further heterogeneity. While majority of ADCs use amide and thioether chemistry to link potent cytotoxic agents to antibodies via endogenous lysine and cysteine residues and maleimide-cysteine conjugation has been used for many clinical stage ADC programs, maleimides have been shown to exhibit some degree of post-conjugation instability. Thus, there is a need for LDCs with an improved stability of the drug-antibody linkage to ensure target specific delivery of a drug and limit off target toxicities.

Such development of improved LDCs typically requires multiple bioanalytical assays. Biotransformations, and drug or drug-linker stability, may be assayed by measuring the concentration of drug that is stably conjugated to the ligand over time, or after exposure to the biological environment using various analytic methods. Such assays require means of releasing the drug or a portion thereof for subsequent measurement. This may be done by enzymatic cleavage. However, some drugs and drug-linkers are not cleavable by enzyme. Therefore, there is a need for alternative means of cleaving drugs and drug-linkers from LDCs, which are suitable for use with appropriate analytic methods for detection and quantitation of released drugs or portions thereof.

### SUMMARY

The present disclosure provides methods of measuring, analyzing and quantifying LDC in a sample, thereby determining the amount of a drug conjugated to a ligand. Specifically, the methods use an LDC comprising an analytic target that can be released from the LDC by treatment with acid, e.g., aqueous trifluoroacetic acid (TFA). Further provided includes the methods of determining the amount, concentration, and stability of an LDC based on the measurement of the analytic target released from the LDC. The method of analyzing an LDC provided herein can be an essential tool for the development of a novel LDC with a better stability and less toxicity.

More specifically, in one aspect, the present invention provides a method of analyzing a ligand-drug conjugate (LDC) in a sample, comprising the step of: (a) providing the sample comprising the LDC, wherein the LDC comprises a ligand and an analytic target, wherein the analytic target comprises a drug molecule or a portion thereof; and (b) contacting the sample with aqueous trifluoroacetic acid (TFA) at a concentration between 1 to 30% (v/v), thereby inducing release of the analytic target from the LDC.

In some embodiments, the method further comprises the steps of: (a) measuring the amount of the analytic target released from the LDC; and (b) determining the concentration of the drug molecule or the portion thereof in the sample using the amount of the released analytic target.

In some embodiments, the step of measuring the amount of the analytic target released from the LDC comprises subjecting the analytic target to liquid chromatography-mass spectrometry (LC-MS). In some embodiments, the step of measuring the amount of the analytic target released from the LDC comprises subjecting the analytic target to liquid chromatography tandem mass spectrometry (LC-MS/MS).

In some embodiments, the method further comprises the steps of: (a) measuring the amount of the ligand in the sample; and (b) determining the concentration of the drug molecule or the portion thereof in the sample by using the measured amount of the ligand.

In some embodiments, the method further comprises the step of collecting the LDC from the sample prior to the step of contacting the sample with aqueous trifluoroacetic acid (TFA). In some embodiments, the step of collecting the LDC is performed by affinity chromatography, size exclusion chromatography, ammonium sulfate precipitation, ion exchange chromatography, immobilized metal chelate chromatography, or immunoprecipitation.

In some embodiments, the step of measuring the amount of the analytic target released from the LDC is performed by using a standard curve of the LDC.

In some embodiments, the method further comprises the steps of: (a) adding to the sample a fixed amount of an internal standard, wherein the internal standard comprises the ligand and a second analytic target, wherein the second analytic target is a labeled derivative of the LDC; (b) contacting the sample with aqueous trifluoroacetic acid (TFA) at a concentration between 1 to 30% (v/v), thereby inducing release of the analytic target from the LDC and the second analytic target from the internal standard; (c) measuring the amount of the second analytic target released from the internal standard; and (d) measuring the amount of the analytic target released from the LDC based on the amount of the second analytic target released from the internal standard.

In some embodiments, the second analytic target has a different molecular weight than the analytic target. In some embodiments, the internal standard comprises an isotopically labeled version of the LDC. In some embodiments, the isotopic label is stable or non-stable. In some embodiments, the isotopic label is deuterium or carbon 13.

In some embodiments, the method further comprises the step of: collecting the LDC and the internal standard from the sample prior to the step of contacting the sample with aqueous trifluoroacetic acid (TFA). In some embodiments, the step of collecting the LDC or the internal standard is performed by affinity chromatography, size exclusion chromatography, ammonium sulfate precipitation, ion exchange chromatography, immobilized metal chelate chromatography, or immunoprecipitation. In some embodiments, the ligand is an antibody or a functional fragment thereof and the LDC or the internal standard are extracted from the sample by contacting the sample with a resin selected from a Protein A resin, a Protein G resin and a Protein L resin.

In some embodiments, the sample is contacted with aqueous trifluoroacetic acid (TFA) at a concentration of 10% (v/v).

In some embodiments, the drug molecule is monomethyl auristatin E (MMAE) or monomethyl auristatin F (MMAF). In some embodiments, the drug molecule is monomethyl auristatin F (MMAF).

In some embodiments, the analytic target comprises a tetra-peptide, Val-Dil-Dap-Phe.

In another aspect, the present invention provides a method of determining stability of the ligand-drug conjugate (LDC), comprising the steps of: (a) obtaining a first sample and a second sample from a single source at different time points after exposure to the LDC; (b) analyzing the LDC in the first sample and the second sample by the method provided herein, thereby determining the amounts of the analytic target released form the LDC in the first sample and the second sample; and (c) determining stability of the LDC by comparing the amounts of the released analytic target in the first sample and the second sample.

In some embodiments, the method further comprises the steps of: (a) measuring the amounts of the ligand in the first sample and the second sample; and (b) determining the ratios of the amount of the released analytic target and the ligand in the first sample and the second sample.

In some embodiments, the sample, the first sample, or the second sample is a biological sample derived from mammalian tissues or aqueous mammalian fluids. In some embodiments, the biological sample is obtained from one of the following: plasma, serum, blood, tissue, tissue biopsy, feces, and urine. In some embodiments, the biological sample is obtained from plasma. In some embodiments, the plasma was treated with the LDC. In some embodiments, the plasma is from a human subject that has been treated with the LDC.

In yet another aspect, the present invention provides a method for quantifying an LDC in a sample, comprising the steps of: (a) providing a sample comprising the LDC, wherein the LDC comprises an analytic target, the analytic target comprising a drug molecule; (b) adding to the sample an internal standard, wherein the internal standard is a labeled derivative of the LDC and comprises a second analytic target; (c) extracting the LDC and the internal standard from the sample; (d) contacting the LDC and the internal standard with aqueous TFA at a concentration between 1 to 30% (v/v), wherein the TFA releases the analytic target from the LDC and the second analytic target from the internal standard; (d) determining the amount of the analytic target released from the LDC and the second analytic target released from the internal standard, wherein the amount of the analytic target released from the LDC correlates with the amount of LDC in the sample.

In some embodiments, the amount of the analytic target released from the LDC is determined by using the amount of the second analytic target released from the internal standard, wherein the amount of analytic target released from the LDC correlates with the concentration of the drug molecule conjugated to an antibody in the LDC in the sample.

In some embodiments, the amount of the analytic target released from the LDC is determined by using a standard curve of the LDC.

In some embodiments, the drug molecule is monomethyl auristatin F (MMAF) or monomethyl auristatin E (MMAE). In some embodiments, the analytic target comprises MMAF or tetra-peptide Val-Dil-Dap-Phe. In some embodiments, the analytic target comprises mcMMAF. In some embodiments, the analytic target and the second analytic target comprises tetra peptide Val-Dil-Dap-Phe and the second analytic target is isotopically labeled with 6 or more carbon and 13 or 6 or more deuterium. In some embodiments, the analytic target and the second analytic target comprises a pegylated linker DPR-PEG-gluc-carbamate-MMAE. In some embodiments, the analytic target and the second analytic target comprises MMAE and the second analytic target is isotopically labeled with 6 or more carbon and 13 or 6 or more deuterium.

In some embodiments, the LDC and the internal standard are contacted with the aqueous TFA concentration at a concentration of 10% v/v.

In one aspect, the present invention provides a kit for determining the amount of an LDC in a sample, comprising: (a) an internal standard for the LDC, wherein the internal standard is a labeled derivative of the LDC, and comprises a drug molecule; and (b) aqueous trifluoroacetic acid TFA for application at a selected concentration between 1 to 30% (v/v). In some embodiments, the internal standard is isotopically labeled.

In another aspect, the present invention provides a kit for determining the amount of an LDC in a sample, comprising: (a) a labeled linker-drug complex and a ligand, wherein the labeled linker-drug complex can be conjugated to the ligand, thereby forming an internal standard; and (b) aqueous trifluoroacetic acid TFA for application at a selected concentration between 1 to 30% (v/v). In some embodiments, the internal standard is isotopically labeled.

### BRIEF DESCRIPTION OF THE DRAWINGS

FIG. 1 provides the ex vivo stability profile of two mAb-mcMMAF ADCs. Citrated rat plasma was spiked with the ADCs, and the samples were analyzed at each time point. ADCs were captured on Protein A affinity resin, and the drug was released using 10% aqueous TFA. The released drug was then quantified by LC-MS/MS. Each time point reflects the percent of the conjugated drug that was observed at to.

FIG. 2 illustrates the change in drug loading over time for an ADC from patient samples. Clinical samples from patients treated with mAb-mcMMAF ADC every 3 weeks (q3w) or every 6 weeks (q6w) were analyzed. After Protein A affinity capture, 10% TFA—mediated release, and drug quantification by LC-MS/MS, the samples were further analyzed for antibody concentration using ELISA. TFA treatment released the tetrapeptide Val-Dil-Dap-Phe, which was quantified by LC-MS/MS. Results are plotted as drugs per antibody over time.

FIG. 3 provides the in vivo stability profile of a mAb-MMAE ADC. The acid release product MMAE was analyzed according to the described method and plotted as amount of conjugated drug over time.

FIG. 4A shows predicted molecular structures with sites selected for conversion to cysteine near the hinge region of the antibody C_{H}2 domain. Sites were first identified on the Fc fragment proximal to the hinge between the Fc and the Fab (left panel). These sites coincide with the CD16 binding sites as shown in the co-crystal structure 1E4K (center panel). Relative orientations of the Fc, Fabs, and CD16 can be seen in the model generated from docking CD16 onto the intact antibody crystal structure 1HZH (right panel). FIG. 4B shows solvent accessibility of conversion sites calculated using 1HZH as a template FIG. 4C provides electrostatic potential calculated for the modeled in silico mutants projected on the molecular surface. These sites showed no consistent trend in either highly acidic or basic elements near the engineered site of conjugation.

FIG. 5 illustrates drug conjugation sites confirmed by proteolysis and mass spectrometry. Wild-type (WT Fc), engineered cysteine antibodies (S239C) and ADCs (S239C+Drug) were digested with endoproteinase GluC (cleavage at position E233 and C-terminal to the hinge disulfide bonds (FIG. 5, left)) followed by subsequent analysis of the Fc fragment using time-of-flight mass spectrometry. When a wild-type ADC is digested, the resulting Fc fragment has a mass of 24,054 Da (top panel) showing no signs of conjugation, consistent with all of the conjugation sites being on the N-terminal side of position 233. Digestion of an S239C antibody results in an Fc fragment with an additional 16 Da in mass, 24,070 Da total, corresponding to the difference in mass between serine and cysteine (center panel). The digestion of a S239C pure 2-loaded ADC results in an Fc fragment with an additional 942 Da in mass, 24,995 Da total, corresponding to the differing masses of serine and cysteine and the addition of the drug linker (bottom panel).

FIG. 6 shows in vivo activity of naked antibody, native 4-loaded ADC and engineered cysteine antibodies (K326C, E269C, A327C, and S239C). Antibodies were tested for activity in a single 10 mg/kg dose 786-0 xenograft experiment. The 2-loaded S239C engineered cysteine outperformed the native 4-loaded and all other engineered cysteine mutant ADCs.

FIGS. 7A-B provides data representing ADC maleimide stability in plasma. FIG. 7A provides a schematic where step 1 shows the reversible Michael addition used to conjugate antibody and drug linker. Step 2 illustrates a potential hydrolysis reaction that stabilizes the conjugate and prevents loss of the drug linker. FIG. 7B shows time course stability of drug-linker conjugate. The data shows loss of the conjugated drug via the retro-Michael reaction during incubation of the ADC with rat plasma. The 2-loaded S239C engineered cysteine is more stable than the native 4-loaded and all other engineered cysteine mutant ADCs. Terminal % drug load relative to t=0 hr for each construct is shown in Table 2.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Definitions

Unless defined otherwise, all technical and scientific terms used herein have the meaning commonly understood by a person skilled in the art to which this invention belongs. As used herein, the following terms have the meanings ascribed to them below.

A “ligand-drug conjugate” or “LDC” refers to a ligand (e.g. an antibody) conjugated to a pharmaceutical agent, e.g. to a cytotoxic or cytostatic drug. “Ligands” include, but are not limited to, polymers, dendrimers, oligonucleotides, proteins, polypeptides, peptides, including cyclic peptides and glycopeptides, or any other cell binding molecule or substance. More specifically, ligands include aptamers (oligonucleotides or peptides), as well as various proteins, such as interferons, lymphokines, knottins, adnectins, anticalins, darpins, avimers, Kunitz domains, and centyrins. Additional ligands include hormones, growth factors, colony-stimulating factors, vitamins, and nutrient transport molecules. Suitable ligands include, for example, antibodies, e.g. full-length antibodies and antigen binding fragments thereof. Antibodies also include bispecific antibodies and multi specific antibodies.

An “antibody-drug conjugate” or “ADC” refers to an antibody, antigen-binding fragment, or engineered variant thereof conjugated to a pharmaceutical agent. Typically, antibody-drug conjugates bind to a target antigen (e.g., CD70) on a cell surface, followed by internalization of the antibody-drug conjugate into the cell and subsequent release of the drug into the cell. The antibody or antigen-binding fragment thereof may be covalently or non-covalently bound to the pharmaceutical agent. In specific embodiments, the drug in LDCs and particularly that in ADCs, is conjugated to the ligand, or more particularly the antibody, through a linker. The linker typically comprises residues resulting from conjugation to the drug and conjugation to the ligand separated by a chemical spacer. The chemical spacer may simply be a hydrocarbon chain, an alkenylene, (e.g., —(CH₂)n-, where n is a selected integer, or n is 2-10), or a heteroalkenylene chain containing one or more oxygens, carbonyls (C═O), sulfurs, or amino groups (e.g., NH or Nalkyl). The linker may be structurally more complex, for example, the linker may be substituted—with a PEG (polyethylene glycol) group, or other hydrophilic group or may contain a cleavable group, e.g, a β-glucuronide that is cleavable by β-glucuronidase, such that cleaving the group, cleaves the linker.

The linker is a chemical species linking the ligand to the drug. Typically, the LDC is formed by two conjugation steps. A precursor to the linker, which is a heterobifunctional species, having two different reactive groups most often separated by a spacer and optionally substituted is most often reacted with the drug molecule to form a linker-drug combination which retains one of the reactive groups. A heterobifunctional linker precursor contains the spacer between the two reactive groups with different reactivity. For example, a heterobifunctional linker precursor may contain an amine-reactive group at one end and a thiol reactive group at the other end. In another more specific example, a heterobifunctional linker precursor may contain a carbonate for reaction with an amine of the drug to form a carbamate. In other more specific examples, a heterobifunctional linker precursor may contain an azide or a N-hydroxysuccinimide ester (NHS ester or a sulfo-NHS ester) for reaction with an amine of the drug to form an amide. Each of such amine reactive groups can be paired in a linker precursor with a maleimide group, which under selected known conditions, is selective for reaction with thiols. After conjugation to the drug, one of the reactive groups remains in the linker-drug combination.

The linker-drug combination retaining the reactive group can then be used as a reagent for conjugation of the drug to the ligand. For example, a ligand conjugation reagent can contain a maleimide group for reaction with thiol groups on a ligand. More generally, the ligand conjugation reagent can contain any appropriate reactive groups for conjugation to groups on the ligand. The reactive groups may react, for example, with amine groups, with carboxylate groups, with thiol groups or with hydroxyl groups.

An “analytic target” refers to a drug or a portion thereof that is released or cleaved from a ligand-drug conjugate, and which is detected or measured (quantitated) by one or more known analytic techniques, e.g. mass spectrometry. The analytic target contains at least the drug or a portion thereof and may in addition contain a portion of the linker. The amount of analytic target is representative of the amount of the ligand-drug conjugate from which it is released or cleaved. More specifically the analytic target is the drug of the LDC or a portion of the drug of the LDC. In specific embodiments, where the drug is an auristatin, the analytic target can be a tetrapeptide released from the drug.

When an internal standard is used, an analytic target can be a drug or a portion thereof that is released or cleaved from the internal standard. In typical embodiments, an analytic target released from an internal standard can be differentiated from an analytic target released from a ligand-drug conjugate, for example, by having a different molecular weight and/or by being labeled.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′)₂, a Fv fragment, a diabody, a single-chain antibody, an scFv fragment, or an scFv-Fc. Genetically, engineered intact antibodies and fragments such as chimeric antibodies, humanized antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen.

The terms “extract”, “extracted”, “extraction”, and “extracting” refer to isolation of an LDC or ADC from a heterogeneous sample comprising several proteins and other molecules. Any appropriate method or material known in the art that can selectively extract an LDC or ADC from a heterogeneous sample, particularly a biological sample, can be employed in the methods herein. Extraction, for example, can include: affinity chromatography, size exclusion chromatography, ammonium sulfate precipitation, ion exchange chromatography, immobilized metal chelate chromatography, and immunoprecipitation.

Binding of LDC or ADC to a resin which contains a species to which the ligand or antibody binds can be used for extraction. Antibody binding proteins can be used for extraction of ADCs. For example, extraction of an ADC from a sample may involve running the sample over a protein A column or contacting the sample with a protein A resin and thereafter removing the resin from the sample in order to capture the antibody, thereby extracting the ADC from the sample. With respect to ADC's, surface proteins protein A, protein G or protein L may be used for extraction. The structural requirements for binding of a given antibody to protein A, protein G or protein L are known in the art and one of ordinary skill in the art can select from among them, the appropriate surface protein for use with a given antibody. Materials useful in extractions using these proteins include resins, e.g., beaded agarose, or magnetic beads, or similar support material to which the protein A, protein G or protein L is covalently immobilized.

The terms “intracellularly cleaved” and “intracellular cleavage” refer to a metabolic process or reaction inside a cell on a ligand-drug conjugate (e.g., an antibody-drug conjugate), whereby the covalent attachment, e. g, the linker between the drug moiety and the ligand unit is broken, resulting in free drug, or other metabolite of the conjugate dissociated from the antibody inside the cell. The cleaved moieties of the drug-linker-ligand conjugate are thus intracellular metabolites.

The terms “release”, “released”, and “releasing” refer to extracellular cleavage of an analytic target from an LDC by the acid-mediated cleavage method described therein. For a given LDC carrying (i.e., conjugated with) a given number of linker-drug combinations, the amount of analytic target released will typically vary with acid concentration (see below) used in the release reaction, the temperature and pressure of the reaction (see below) and the reaction time employed. For consistency of results from sample to sample, the same acid concentration and reaction conditions should be employed. Treatment with acid as described herein need not release all analytic target from the LDC. All that is needed is to release an amount of analytic target that is sufficient for obtaining an accurate and precise measurement of the analytic target in view of the analytic method employed.

The terms “contact”, “contacted”, and “contacting” refer to adding acid or reagent to a sample, which may be a test sample or a control sample(including biological samples), so that the components of the sample are made available to the acid or reagent, and a reaction can thus occur. The reaction associated with acid addition in the method herein is release of an analytic target from an LDC or more specifically an ADC.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. The term includes radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁰C, and radioactive isotopes of Lu), chemotherapeutic agents, and toxins such as small molecule toxins or enzymatically active toxins of bacterial, fungal, plant or animal origin, including synthetic analogs and derivatives thereof. In certain embodiments, a cytotoxic agent is conjugated to an antibody or administered in combination with an antibody. Suitable cytotoxic agents are described further herein.

“Cytotoxic activity” refers to a cell-killing, a cytostatic or an anti-proliferative effect of a ligand-drug conjugate compound or an intracellular metabolite of a ligand-drug conjugate. Cytotoxic activity may be expressed as the IC₅₀ value, which is the concentration (molar or mass) per unit volume at which half the cells survive.

The term “patient” or “subject” includes human and other mammalian subjects such as non-human primates, rabbits, rats, mice, and the like and transgenic species thereof, that receive either prophylactic or therapeutic treatment.

The term “standard curve” or “calibration curve” refers to a graph used as a quantitative research technique. To generate the standard curve, multiple samples with known properties are measured and graphed, which then allows the same properties to be determined for unknown samples by interpolation on the graph. The samples with known properties are the standards, and the graph is the standard curve. Standard curves are of particular use when measuring the amount or concentration of an analyte in a sample that may contain an unknown amount of the analyte. The use of a standard curve alone represents the use of an external standard. As is understood in the art, the standard curve of a given analyte (i.e., the LDC) to be quantitated should generally span the concentration range of the analyte expected in the samples. Again as is understood in the art, samples used for preparing the standard curve are processed by the same steps as test samples and any control samples in which the analyte is to be measured. A standard curve can also be employed in combination with the use of an internal standard. In this case, a constant (or fixed) amount of the internal standard is added to each sample used to generate the standard curve of known analyte concentrations. The same constant amount of internal standard is added to each test sample and to any blanks or control samples. The details of use of standard curves (calibration curves) as an external standard and a combination of the use of a standard curve with addition of internal standard for quantitation of analytes by analytic methods, including MS, LC-MS and LC-MS/MS methods, is well known in the art. One of ordinary skill in the art understands how to use such analytic methods in the determination of concentrations of analytes in a variety of samples, including biological samples as discussed herein.

An “internal standard” is a chemical species that behaves in a selected assay similarly to the chemical species to be quantitated (i.e., LDC), but which is distinguishable from that chemical species in the analytic method being used. Typically, the internal standard is labeled to distinguish it from the chemical species to be quantitated, but the label employed does not significantly differentially affect its behavior compared to that of the chemical species to be quantitated. Preferably, anything that affects the measurement of the chemical species to be quantitated (e.g., analyte peak area) will also affect the measurement of the internal standard similarly. The ratio of the measurements of the chemical species to be quantitated and its internal standard preferably exhibits less variability than the measurement of the chemical species in a test sample. For use in mass spectrometry methods, the internal standard has a molecular weight that is different from the chemical species to be quantitated.

Most often labeling with stable isotopes, such as deuterium (²H) and carbon 13 (¹³C) is employed. Labeling must allow separate measurement of analyte and internal standard. Preferably, an isotopically labeled internal standard differs in molecular weight from the chemical species to be quantitated by at least 3 amu (i.e., labeling with 3 or more ²H or ¹³C). More specifically, labeling results in a difference in molecular weight of 6 amu or more. Internal standards can also be surrogates of the chemical species to be quantitated. Surrogate internal standards differ structurally from the chemical species to be quantitated by substitution of an atom or chemical group by a different group, for example the substitution of a methyl group or other small alkyl for a hydrogen, or the substitution of a halogen, e.g., a fluorine, for a hydrogen. Such surrogates may be of particular use where it is not possible to readily obtain an isotopically labeled internal standard.

The terms “determine”, “determined”, and “determining” refer to the ascertaining of the concentration or amount of a particular analyte based on a measurement of the amount of an analytic target and the known amounts of one or more correlative factors. As is understood in the art, an analyte concentration can be combined with the results of other measurements to determine other structural and physical properties of an analyte.

When trade names are used herein, the trade name includes the product formulation, the generic drug, and the active pharmaceutical ingredient(s) of the trade name product, unless otherwise indicated by context.

### Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### Assay for Analyzing a Ligand-Drug Conjugate (LDC)

In one aspect, the present invention provides a method of analyzing a ligand-drug conjugate (LDC) in a sample, comprising the step of: (a) providing the sample comprising the LDC, wherein the LDC comprises a ligand and an analytic target, wherein the analytic target comprises a drug molecule or a portion thereof; (b) contacting the sample with aqueous trifluoroacetic acid (TFA) at a concentration between 1 to 30% (v/v), thereby inducing release of the analytic target from the LDC. In some embodiments, the method can comprise the steps of (a) providing a sample comprising the LDC, wherein the LDC comprises an analytic target, the analytic target comprising a drug molecule; (b) adding to the sample an internal standard, wherein the internal standard is a labeled derivative of the LDC and comprises a second analytic target; (c) extracting the LDC and the internal standard from the sample; (d) contacting the LDC and the internal standard with aqueous TFA at a concentration between 1 to 30% (v/v), wherein the TFA releases the analytic target from the LDC and the second analytic target from the internal standard; (e) determining the amount of the analytic target released from the LDC and the second analytic target released from the internal standard, wherein the amount of the analytic target released from the LDC correlates with the amount of LDC in the sample.

A Sample Comprising Ligand-Drug Conjugate (LDC)

The present invention provides a method of analyzing a ligand-drug conjugate (LDC) in a sample. An LDC is a complex comprising a ligand and an analytic target. The analytic target comprises a drug molecule or a portion thereof. Various samples comprising an LDC or suspected to comprise an LDC can be subject to analysis using a method provided herein. In particular biological sample can be analyzed.

Sample

An LDC in a biological or non-biological sample can be analyzed by the methods provided herein. In preferred embodiments, the sample is a biological sample derived from a mammalian subject. Specifically, in some embodiments, the biological sample is obtained from one of the following: plasma, serum, blood, tissue, tissue biopsy, feces, and urine.

In some embodiments, the sample is a biological sample contacted with an LDC in vivo. For example, the sample can be a biological sample derived from a subject exposed to an LDC. In some embodiments, the sample is obtained at a specific time point after administration of an LDC. In some embodiments, the sample is obtained at multiple time points after administration of an LDC. In some embodiments, the sample is obtained before administration of an LDC.

In some embodiments, the sample is a biological sample contacted with an LDC ex vitro. In some embodiments, the sample is contacted with an LDC for a specific time period. In some embodiments, a plurality of samples contacted with LDC for different periods are subject to analysis. In some embodiments, the sample is obtained before exposure to an LDC.

Ligand-Drug Conjugate (LDC)

Ligand

In some embodiments, the ligand is a protein having specific affinity to a target molecule. In some embodiments, the ligand is an antibody. Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

The antibody can be a functionally active fragment, derivative or analog of an antibody that immunospecifically binds to target cells (e.g., cancer cell antigens, viral antigens, or microbial antigens) or other antibodies bound to tumor cells or matrix. In this regard, “functionally active” means that the fragment, derivative or analog is able to elicit anti-idiotype antibodies that recognize the same antigen as the antibody from which the fragment, derivative or analog is derived. Specifically, in an exemplary embodiment the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIA core assay) (See, e.g., Kabat et al., 1991, Sequences of Proteins of Immunological Interest, Fifth Edition, National Institute of Health, Bethesda, Md.; Kabat E et al., 1980, J. Immunology 125(3):961-969).

Other useful antibodies include fragments of antibodies such as, but not limited to, F(ab′)2 fragments, Fab fragments, Fvs, single chain antibodies, diabodies, tribodies, tetrabodies, scFv, scFv-Fv, or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from murine monoclonal and human immunoglobulin constant regions. (See, e.g., U.S. Pat. Nos. 4,816,567; and 4,816,397, each of which is incorporated herein by reference in its entirety.) Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety.) Such chimeric and humanized monoclonal antibodies can be produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060; each of which is incorporated herein by reference in its entirety.

Completely human antibodies are particularly desirable and can be produced using transgenic mice that are incapable of expressing endogenous immunoglobulin heavy and light chains genes, but which can express human heavy and light chain genes.

Antibodies include analogs and derivatives that are either modified, i.e., by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

Antibodies can have modifications (e.g., substitutions, deletions or additions) in amino acid residues that interact with Fc receptors. In particular, antibodies can have modifications in amino acid residues identified as involved in the interaction between the anti-Fc domain and the FcRn receptor (see, e.g., International Publication No. WO 97/34631, which is incorporated herein by reference in its entirety).

Antibodies immunospecific for a cancer cell antigen can be obtained commercially or produced by any method known to one of skill in the art such as, e.g., chemical synthesis or recombinant expression techniques. The nucleotide sequences encoding antibodies immunospecific for a cancer cell antigen can be obtained, e.g., from the GenBank database or a database like it, the literature publications, or by routine cloning and sequencing.

In certain embodiments, useful antibodies can bind to a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD2O, CD22, CD28, CD30, CD70, CD79, CD90, CD152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d, CD49e, CD49f, CD103, and CD104. Non-limiting examples of suitable lectins are C-type, S-type, and I-type lectin.

In some embodiments, the ligand is a receptor ligand. The receptor ligand can have a binding partner that is enriched in a specific cell type, tissue or organ. The ligand can be a naturally occurring agonist or antagonist of a receptor, or a synthetic molecule that has an affinity to the receptor. The receptor ligand can be a protein, nucleic acid or other receptor ligand such as a peptide, vitamin, and carbohydrate. In one embodiment, the ligand is folate that has affinity to a folate receptor.

In some embodiments, the ligand is a targeting moiety that has been used and developed for targeting a drug to a target organ or tissue. Such site-specific ligands known in the art can be used and adopted in the method provided herein.

Drug

The drug of the LDC can be any cytotoxic, cytostatic or immunosuppressive drug also referred to herein as a cytotoxic, cytostatic or immunosuppressive agent. The drug has a functional group, such as an amino, alkyl amino group or carboxylate that can form a bond with an appropriate reactive group of a reagent precursor containing the linker, such as an amine group, a carboxylic acid group, a sulfhydryl group, a hydroxyl group or an aldehyde or ketone group. In an embodiment, the drug is conjugated to a linker to generate an amide or a carbamate. In an embodiment, the drug is conjugated to a linker by an amide bond. In an embodiment, the drug contains a single amide bond. In an embodiment, the drug is conjugated to the linker by a carbamate and the drug contains an amide bond. In specific embodiments, TFA treatment, releases the drug or a portion thereof by cleavage of the amide bond to the linker or an internal amide bond in the drug.

Useful classes of cytotoxic or immunosuppressive agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like. Particularly useful classes of cytotoxic agents include, for example, DNA minor groove binders, DNA alkylating agents, and tubulin inhibitors. Exemplary cytotoxic agents include, for example, auristatins, camptothecins, duocarmycins, etoposides, maytansines and maytansinoids (e.g., DM1 and DM4), taxanes, benzodiazepines (e.g., pyrrolo[1,4]benzodiazepines (PBDs), indolinobenzodiazepines, and oxazolidinobenzodiazepines) and vinca alkaloids. Select benzodiazepine containing drugs are described in WO 2010/091150, WO 2012/112708, WO 2007/085930, and WO 2011/023883.

In an exemplary embodiment, the drug is a peptidic drug containing one or more, two or more, three or more or four or more amino acid groups. In an exemplary embodiment, the drug is a peptidic drug containing an N-terminal, N-methylated amino acid group. In a further exemplary embodiment, the drug is a peptidic drug having an N-terminal, N-methylated amino acid with an alkyl side group. In a further exemplary embodiment, the drug is a peptidic drug having an N-terminal, N-methylated alanaine, N-methylated isoleucine, N-methylated leucine or N-methylated valine. In a further exemplary embodiment, the drug is a peptidic drug having an N-terminal, N-methylated valine.

In a preferred embodiment, the drug is an auristatin. Auristatins include, but are not limited to, AE, AFP, AEB, AEVB, MMAF, and MMAE. The synthesis and structure of auristatins are described in U.S. Patent Application Publication Nos. 2003-0083263, 2005-0238649 2005-0009751, 2009-0111756, and 2011-0020343; International Patent Publication No. WO 04/010957, International Patent Publication No. WO 02/088172, and U.S. Pat. Nos. 7,659,241 and 8,343,928; each of which is incorporated by reference herein in its entirety and for all purposes. Exemplary auristatins of the present invention bind tubulin and exert a cytotoxic or cytostatic effect on the desired cell line. In an embodiment, exemplary auristatins contain an N-terminal, N-methylated amino acid. More specifically, exemplary auristatins contain an N-terminal N, N-methylated amino acid with an alkyl side chain, such as alanine, isoleucine, leucine, or valine. Yet more specifically, exemplary auristatins contain an N-terminal, N-methylated valine.

Other individual cytotoxic or immunosuppressive agents include, for example, an androgen, anthramycin (AMC), asparaginase, 5-azacytidine, azathioprine, bleomycin, busulfan, buthionine sulfoximine, calicheamicin, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, etoposide, an estrogen, 5-fluordeoxyuridine, 5-fluorouracil, gemcitabine, gramicidin D, hydroxyurea, idarubicin, ifosfamide, irinotecan, lomustine (CCNU), maytansine, mechlorethamine, melphalan, 6-mercaptopurine, methotrexate, mithramycin, mitomycin C, mitoxantrone, nitroimidazole, paclitaxel, palytoxin, plicamycin, procarbizine, rhizoxin, streptozotocin, tenoposide, 6-thioguanine, thioTEPA, topotecan, vinblastine, vincristine, vinorelbine, VP-16 and VM-26.

Suitable cytotoxic agents also include DNA minor groove binders (e.g., enediynes and lexitropsins, a CBI compound; see also U.S. Pat. No. 6,130,237), duocarmycins (see U.S. Publication No. 20060024317), taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone.

Examples of anti-tubulin agents include, but are not limited to, taxanes (e.g., Taxol® (paclitaxel), Taxotere® (docetaxel)), T67 (Tularik) and vinca alkyloids (e.g., vincristine, vinblastine, vindesine, and vinorelbine). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. Maytansine and maytansinoid are another group of anti-tubulin agents. (ImmunoGen, Inc.; see also Chari et al., 1992, Cancer Res. 52:127-131 and U.S. Pat. No. 8,163,888).

Exemplary auristatin drugs have the following formula or a pharmaceutically acceptable salt thereof wherein the wavy line indicates site of attachment to the linker:

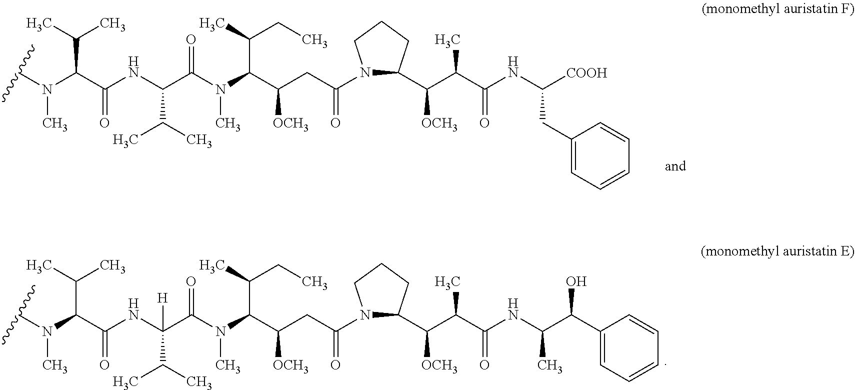 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C ; *N(C)[C@H](C(=O)N[C@@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Alternative auristatin drugs for conjugation to a ligand through a linker have the following formula or a pharmaceutically acceptable salt thereof, where the wavy line indicates the site of attachment to the linker:

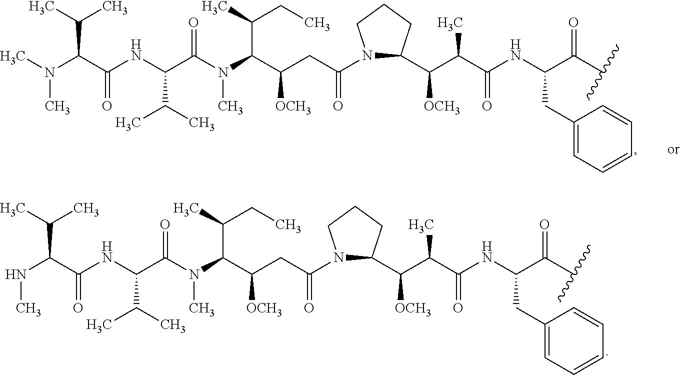 [SMILES: *C(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C ; *C(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@@H](NC)C(C)C)C(C)C]

Additional cytotoxic compounds useful for the preparation of LDCs and particularly useful for the preparation of ADCs are those described in U.S. Pat. No. 6,884,869, which is incorporated by reference herein in its entirety, particularly for descriptions of cytotoxic compounds. Additional description therein describes preparation of drug conjugates with the cyctotoxic compounds described.

Linker

General procedures for linking a drug to linkers are known in the art. See, for example, U.S. Pat. Nos. 8,163,888, 7,659,241, 7,498,298, U.S. Publication No. US20110256157 and International Application Nos. WO2011023883, and WO2005112919.

The linker can be cleavable under intracellular conditions, such that cleavage of the linker releases the therapeutic agent from the ligand in the intracellular environment (e.g., within a lysosome or endosome or caveolea). The linker can be, e.g., a peptidyl linker that is cleaved by an intracellular peptidase or protease enzyme, including a lysosomal or endosomal protease. Intracellular cleaving agents can include cathepsins B and D and plasmin (see, e.g., Dubowchik and Walker, Pharm. Therapeutics 83:67-123, 1999). For example, a peptidyl linker that is cleavable by the thiol-dependent protease cathepsin-B, which is highly expressed in cancerous tissue, can be used (e.g., a linker comprising a Phe-Leu or a Val-Cit peptide). The linker can also be a carbohydrate linker, including a sugar linker that is cleaved by an intracellular glycosidase (e.g., a glucuronide linker cleavable by a glucuronidase).

The linker also can be a non-cleavable linker, such as a maleimido-alkylene- or maleimide-aryl linker that is attached to the ligand via a sulfur (thiol) and released by proteolytic degradation of the antibody.

An antibody can be conjugated to one or more linker via any appropriate reactive group, e.g., via an amine group (for example, an N-terminal amino group or an amine group of an amino acid side group, such as lysine), a thiol group (—SH, for example, that of a cysteine residue), a carboxylate (for example, a C-terminal carboxylate, or that of an amino acid side chain, such as glutamic acid) or a hydroxyl group (for example of a serine residue), of the antibody.

In exemplary ADCs, monomethyl auristatin E is conjugated through a protease cleavable peptide linker to an antibody, monomethyl auristatin F is conjugated to an antibody through the linker maleimidocaproic acid (mc). The linker may, in addition, contain chemical groups that modulate solubility or pharmacokinetics. For example, an exemplary linker is pegylated. Specific exemplary linker-drug combinations are:

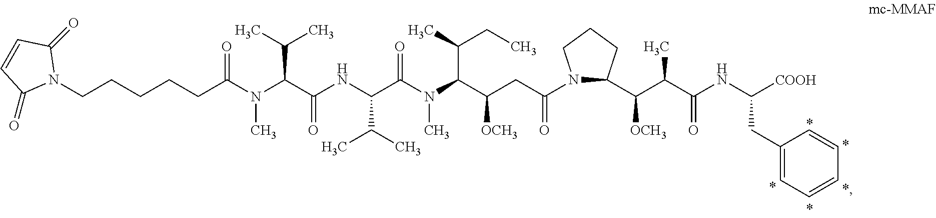 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

wherein me maleimide group of the linker can react with thiol groups of a ligand and particularly of an antibody; or

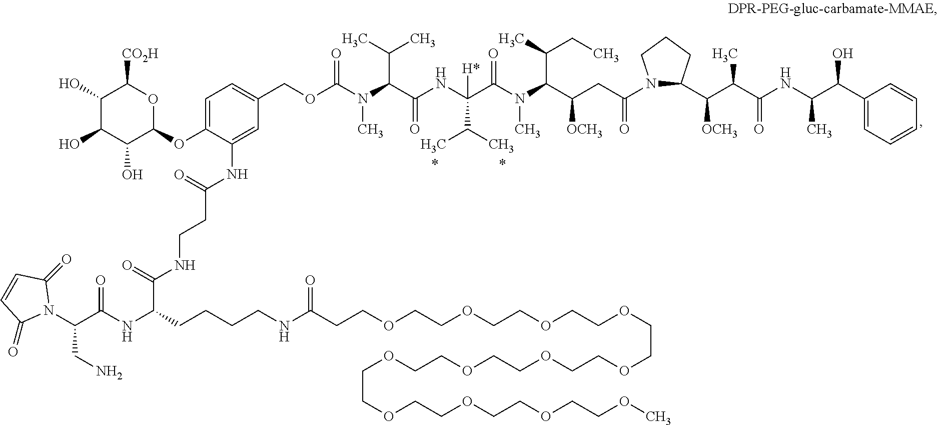

wherein the linker is pegylated and contains a glucuronic acid (cleavable by glucoruonidase) and wherein the maleimide group of the linker can react with thiol groups of a ligand. In LDCs containing the above linker-drug combinations, treatment with acid as described herein releases the tetra peptide Val-Dil-Dap-Phe (where Dap is dolaproline) from mc-MMAF, and the entire drug MMAE from DPR-PEG-gluc-carbamate-MMAE. Internal standards for LDCs and ADCs can be prepared by labeling of such linker-drug combinations, wherein the label is released on treatment with acid as described herein. Exemplary internal standards for LDC and ADC conjugated to mc-MMAF, include those that are deuterated or labeled with ¹³C in the tetrapeptide released. Exemplary internal standards for LDC and ADC conjugated to mc-MMAF, include those that are deuterated or labeled with ¹³C in the MMAE released. In the above structures, sites for possible ¹³C labeling or deuterium labeling are shown by “*.”

Quantitation methods herein generally employ the release of a fragment of a LDC, designated as an analytic target herein, which represents the entire LDC, and which analytic target is quantitated. Quantitation of the analytic target allows one to measure the amount of analytic target released, the amount of analytic target in the LDC in a sample and/or the amount of LDC in a sample. In some determinations, it is necessary to know or to determine, by appropriate known methods, the amount of ligand in a sample or to know or to determine, by appropriate methods, the number (or average number) of drug molecules conjugated to a given LDC. More specifically, the analytic target herein is the drug molecule of the LDC or a portion of the drug molecule of the LDC. Drugs are conjugated to the ligand in an LDC by a linker species, so an analytic target may also include a portion of or the entire linker in addition to the drug or portion thereof. In specific embodiments, herein the analytic target is the drug conjugated to the LDC. In specific embodiments, herein the analytic target is a portion of the drug conjugated to the LDC. In specific embodiments herein, the drug is a peptide or derivative thereof and the analytic target is the peptide drug or a peptide portion of the peptide drug. In specific embodiments, where the drug is a peptide or derivative thereof, the analytic target is a dipeptide or derivative thereof, a tripeptide or derivative thereof, or a tetrapeptide or derivative thereof.

Cleavage Mediated by Trifluoroacetic Acid (TFA)

The method of the present invention comprises the step of contacting a sample with aqueous trifluoroacetic acid (TFA) at a concentration between 1 to 30% (v/v), to induce release of an analytic target from LDC. Solutions of TFA in acetonitrile can also be employed.

The TFA concentration employed can be 1-20%, 1-10%, 2.5-30%, 2.5-20%, 2.5-10%, 5-15%, 7-13%, 9-11%, or 9.5 to 10.5%, v/v with all ranges inclusive. The TFA concentration is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, all % v/v. In a preferred embodiment, the TFA is 10% (v/v).

The TFA concentration may be a result of dilution of 100% TFA in water, sample mixture, or any other acceptable solvent. The TFA may be diluted before it is added to the sample, or diluted in the sample mixture itself.

The TFA reaction may be performed under variable time and temperature conditions. For example, the reaction may be performed at between 20 and 80° C., such as about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C.

The TFA reaction is typically performed at ambient pressure. It will be apparent to one of ordinary skill in the art that the pressure of a reaction may be varied in such a reaction without significant detriment. It will be appreciated that a change in pressure may require a change in temperature. A reaction conducted at a higher pressure may permit a lower reaction temperature to be used. It will be appreciated that concentration of acid, time of reaction and the reaction temperature may be varied within ranges described herein along with the pressure of the reaction to achieve a desired level of release of analytic target.

The reaction may be performed for a period of about 12-24 hours, 10-20 hours, or 15-17 hours. However, any combination of acid concentration, temperature, time, and pressure that allows the selected analytical method to give a measurement of the desired accuracy and precision may be used. As noted elsewhere, for consistency of results in a given experiment or quantitation, the reaction conditions used should be the same for all test samples (unknowns), all controls and all calibration samples for a given experiment or quantitation. In an exemplary embodiment the cleavage reaction is performed using TFA 10%, at 70° C., and at ambient pressure for a period of about 16 hours.

Other acids may be used in the disclosed methods, such as, but not limited to other fluorinated acids, organic or mineral acids. Specific alternative acids include trifluoromethane sulfonic acid. Acids that are volatile are generally preferred over mineral acids, such as HCl.

Measurement of Analytic Target

In some embodiments, the method further involves the step of measuring an analytic target in a sample. An analytic method appropriate for quantitation of the analytic target in the concentration range that is expected to be encountered in samples can be used.

In some embodiments, an LDC or an internal standard is extracted from the sample prior to the measurement of the analytic target. The analytic target can be collected by affinity chromatography, size exclusion chromatography, ammonium sulfate precipitation, ion exchange chromatography, immobilized metal chelate chromatography, or immunoprecipitation. In some embodiments, an LDC or an internal standard includes an antibody or a functional fragment as a ligand. In those cases, the LDC or the internal standard can be collected by contacting the sample with a resin selected from a Protein A resin, a Protein G resin and a Protein L resin.

In some embodiments, the analytic target is detected and quantified using liquid chromatograph/mass spectrometry (LC/MS) methods. More specifically, tandem mass spectrometry (MS/MS) methods are employed. In MS/MS methods, one or more fragment ions of a selected parent ion of the analytic target are monitored. A parent ion of the analytic target is selected as known in the art in a first MS step and that parent ion is subjected to fragmentation, typically collision-induced fragmentation, to generate one or more fragment ions each of which can be quantitated by measurement, for example, of the ion current associated with each fragment to generate ion current peaks as a function of mass (m/z). Integrated peak areas of a fragment can be measured for quantitation of the chemical species from which the parent ion and one or more fragment ions thereof derive. In application to measurement of analytic target herein, the one or more fragments derive from the parent ion of the released analytic target.

Any MS/MS method can be employed for quantitation of analytic targets herein, but methods employing a triple quadrupole or a quadrupole-ion trap are more typically employed. Mass spectrometers used in the methods herein can be operated to monitor the entire mass spectrum of a sample, or more typically a selected portion thereof of interest. Particularly in MS/MS methods, the signal (e.g., ion current) from one or more fragment ions of a selected parent ion may be monitored. Selected reaction monitoring (SRM) operation can be used in which a single fragment ion generated from a selected parent ion is monitored. Alternatively, multiple reaction monitoring (MRM) operation can be used in which more than one fragment ion generated from a selected parent ion is monitored. The use of the term fragment ion relates to ions generated in MS/MS by the dissociation or fragmentation of a selected ion. It will be appreciated that methods are known in the art and used for quantitation of analytes that involve reacting selected parent ions to more generally generate product ions which include fragment ions as well as other product ions that are not fragment ions. MS/MS methods which generate all such product ions can be analogously employed in the methods herein.

In some embodiments, a liquid chromatography method appropriate for use in quantitation of analytic targets in various samples is used.

In some embodiments, the method involves use of standard curves (calibration curves) as an external standard and a combination of the use of a standard curve with addition of internal standard for quantitation of analytes by MS, LC-MS and LC-MS/MS methods. In some embodiments, the standard curves can be used to determine concentrations of analytes in a variety of samples, including biological samples as discussed herein. Specifically, the amounts of analytes from internal standard can be used to determine the amounts of analytes from an LDC. In particular embodiments, the amounts of analytes from internal standard are used to generate standard curves for use in determination of amounts of analytes from an LDC. In these embodiments, analytes from internal standard and analytes from an LDC can be differentiated by labeling.

Concentration Assay

In some embodiments, the method further comprises the step of determining the concentration of an LDC in a sample. The present invention also provides a method for determining, in a sample, the concentration of a drug that is conjugated to a ligand in an LDC.

The quantitation analysis preferably includes calibration within the assay. A standard curve can be generated, for example, by preparing a series of at least 6 samples with increasing concentrations of LDC. The internal standard is added to the standard curve samples, which are then processed by the protein A and LC-MS/MS methods described above. The peak area for each standard is divided by the peak area obtained for the internal standard, and the resultant peak area ratios are plotted as a function of standard concentrations. In some embodiments, at least 6 data points are fitted to a curve using, for example, linear regression analysis.

Stability Assay

In some embodiments, the method is used to determine stability of an LDC.

In an exemplary assay, the LDC is placed in sterile plasma and incubated at 37° C. At the beginning of the incubation and at varying timepoints from 1 hour to 1 week or longer, an aliquot is removed and at frozen at −80° C. Upon completion of the timepoints, the samples are subjected to a protein purification method that will specifically extract the ligand and conjugated drug. For example, an antibody-drug conjugate may be passed over a protein A affinity resin to capture the antibody, and subsequently the resin is washed with buffer. After capture of the ligand-drug conjugate, the drug is released from the captured ligand by treatment with 1-30% (v/v) trifluoroacetic acid. The released drug can then be quantified by standard LC-MS methodology, and the quantity of drug measured at each timepoint divided by the quantity of drug measured for the pre-incubation aliquot can be used to determine the percentage of drug remaining conjugated to the ligand at each timepoint. The precision of this assay can be improved by including an internal standard ligand-drug conjugate which is prepared using an isotopically labeled version of the same drug-linker, such that the drug which is released from it can be detected independently in the LC-MS assay from the drug released from the test drug-linker by virtue of its mass difference. This isotopically labeled internal standard ligand-drug conjugate is added to each sample in equal amounts immediately prior to the ligand capture step (e.g. protein A). The quantitation of the drug or a portion of the drug released from the test LDC is then performed using the internal standard by conventional liquid chromatography—mass spectrometry (LC-MS/MS) techniques. Mass spectrometry techniques for use in pharmacokinetics assays are known in the art. (See, for example, Want et al., Spectroscopy 17:681-691 (2003); Okeley et al., Clin Cancer Res. 16: 888-897 (2010); Singh et al., DMD (2017); Alley et al., Bioconjugate Chem. 19:759-765 (2008).).

In other embodiments, an LDC is administered to a subject and samples are obtained from the subject at different time points after administration of the LDC. The plurality of samples are subject to the methods provided herein for measurement of analytic target from the LDC. In some embodiments, internal standard is administered together with the LDC. Amounts of LDC in the samples can be compared and used to determine stability of the LDC over time.

In some embodiments, an LDC is added to a sample ex vivo. Samples are collected after various time points after addition of the LDC. The plurality of samples are subject to the methods provided herein for measurement of analytic target from the LDC. In some embodiments, internal standard is added to the sample together with the LDC. Amounts of LDC in the samples can be compared and used to determine stability of the LDC ex vivo over time.

Other Assays

The method provided herein can be used to determine average number of drugs per ligand. For example, average number of drugs per ligand can be measured by dividing the concentration of ligand conjugated drug, obtained by the methods described herein, by the concentration of ligand.

In other embodiments, the acid-mediated cleavage methods and related analytic methods described herein can be used in a variety of experiments that rely on the determination of the amount of LDC in a sample or determining the amount of drug conjugated to an LDC. The methods herein can, for example, be used for determining release kinetics of drugs from LDCs in the context of developing clinical agents for treatments of diseases or disorders. The methods herein can also be used for studying the pharmokinetics of an LDC. The methods herein can be used to assess the use of LDCs in clinical applications.

### Kit

In another aspect, a kit for measurement of LDC in a sample or for measurement of the amount of drug conjugated to an LDC is provided. A kit comprises one or more chemical and typically more than one chemical component useful for carrying out an assay as described herein. In a kit, the different chemical components are typically provided in selected amounts in separate containers packaged together and optionally including instructions for carrying out the assay. The amounts of chemical components in a given kit are typically provided in selected amounts to carry out a selected number of assays for each kit. For example, each kit can be designed to carry out one assay and thus is provided with a sufficient amount of the chemical species to carry out all steps in a given assay. Kits are optionally also provided with reagents or solvents needed for carrying out an assay. Kits can be provided, for example, with reagents for extracting a given LDC or a class of LDC from samples. In an embodiment, kits herein comprise an appropriately labeled internal standard for any given LDC, including any ADC. The internal standard of the kit can be an isotopically labeled LDC, where the label is positioned in the drug. Such kits may also contains unlabeled LDC for preparation of standard curves.

In another embodiment, kits comprise a reagent comprising a labeled linker-drug combination containing a reactive group for conjugating the linker and drug to any selected ligand, including any selected antibody. More specifically, the reagent is labeled in the drug or a portion thereof so that on release of analytic target the label is released with the analytic target. The kit optionally further contains reagents or solvent for carrying out a conjugation with a selected ligand or antibody. A kit may also contain unlabeled linker-drug reagent for preparation of unlabeled LDC. The kit may further contain unlabeled or labeled analytic target, e.g., the drug or the portion of the drug released by acid treatment. In a specific embodiment, a kit contains an isotopically labeled mc-MMAF or an isotopically labeled DPR-PEG-gluc-carbamate-MMAE for conjugation to any selected ligand or antibody to serve as an internal standard for measurement of L-mc-MMAF or L-DPR-PEG-gluc-carbamate-MMAE. Such kits can be used as research aids for development of LDCs suitable for clinical use. Such kits can also be employed in clinical application where there is a need to monitor LDC or LDC drug loading in a patient.

In some embodiments, a kit may comprise a pair of reagents for conjugating the linker-drug combination, and the ligand, in separate packaging, as well as the reagents necessary for a single conjugation reaction. The kit may optionally include solvent or buffer for carrying out reactions and instructions for use. Methods for conjugation of ligands and drug-linkers are known in the art. (See, for example, Lyon et al., Methods in Enzymology, vol. 52, pgs. 123-138, 2012; Sun et al., Bioconjugate Chem. 16:1282-1290, 2005.)ed internal standards and reagents are isotopically labeled with either stable or unstable isotopes. Stable isotopes include, but are not limited to, 2H, ¹³C and ¹⁵N. Radioactive or unstable isotopes include, but are not limited to, ³H, ¹⁴C and ¹²N.

Alternatively, an internal standard may be distinguished from the LDC by a structural modification that confers a different molecular weight, but is not isotopically labeled. For example, an internal standard may comprise a methyl group or a halogen instead of hydrogen at a position in the analytic target. This would, in effect, change the molecular weight, but not substantially change how the internal standard reacts with the TFA. As is appreciated in the art any internal standard for a given analyte used must be assessed to ensure that it behaves as the analyte in a given analytic method.

### EXAMPLES

The following examples are provided by way of illustration not limitation.

### Example 1: Assay Methods

Preparation of Experimental Samples, Calibrators, and Internal Standard (IS)
1. Dilutions of ADC calibrators were prepared in sample matrix (e.g. buffer, plasma, etc.) at the following concentrations of antibody-conjugated drug (ADC):
   a. 8 point calibration curve: 10 μM, 4 μM, 1.6 μM, 640 nM, 256 nM, 102.4 nM, 41 nM, 16.4 nM ADC equivalents
   b. a blank was included (no ADC, sample matrix only).
2. Dilution of ADC internal standard (“IS”) was prepared in sample matrix at a single concentration of 500 nM ADC equivalents.
3. A fixed volume of ADC IS was combined with a fixed volume of each calibrator or unknown sample for a final volume ranging between 250 μl-1000 μl.

The nominal concentrations of the ADC calibrators and IS (in Steps 1 and 2) and the final volume after mixing the IS with ADC calibrators and samples (in Step 3) changes from experiment to experiment; these values are also dependent on the ADC analyzed; this method accommodates a broad range of applications.

Preparation of a 96-Well Filter Plate

Protein A agarose MabSelect (GE Healthcare) was equilibrated in buffer (PBS, pH 7.4) at a slurry ratio of 1 part agarose resin to 3 parts buffer.

800 μl of the slurry (200 μl resin) was added to a filter plate and centrifuged at 1250×g for 5 minutes at 4° C. to remove the aqueous phase.

96-well polypropylene 2 ml dilution blocks were used to collect buffer, sample and calibrator flow through, washes, and elution volumes for each centrifugation step from this point onward.

Sample Capture and Elution

1. 200 μl ADC calibrator (+IS) and experimental samples (+IS) were added to 200 μl Protein A resin and shaken (1 h, 4° C., ˜1000 rpm).

2. The plate was centrifuged at 2000×g for 5 minutes at 4° C. to remove the sample matrix.

3. Wash buffer was added (1×PBS, pH 7.4; 200-400 μl) and centrifuged at 2000×g for 5 minutes at 4° C. to complete removal of sample matrix.

4. The wash step was preformed 1-3 times before elution.

5. To elute ADC from resin, 200 μl of IgG elution buffer (Thermo Scientific) was added and the plate was placed on a shaker (1 h, 4° C., 1000 rpm).

6. The plate was centrifuged at 2000×g for 5 minutes at 4° C. to elute the ADC/IS.

7. Steps 4 and 5 were repeated to complete elution of the ADC/IS from the resin. The combined final volume of eluted ADC/IS was 400 μl.

Sample Processing

1. ADC/IS calibrators and samples (in IgG elution buffer) were evaporated under N₂ gas at 60° C. for 4 hours or until plate was dry.

2. 400 μl of 10% triflouroacetic acid (TFA) (v/v)(diluted in water) was added and the plate was sealed with a Teflon™-coated silicone plate mat.

3. The sealed plate was placed into a jacketed Thermomixer and incubated overnight (˜16 h at 70° C.; ˜600-800 rpm).

4. The plate was centrifuged at 2000×g for 5 minutes at 4° C. to spin down condensation.

5. The ADC/IS calibrators and samples (in 10% TFA, v/v) were evaporated under N₂ gas at 40° C. for 4 hours or until plate was dry.

6. 500 μl of ice cold 100% MeOH was added, the plate was covered with a plate sealer, and placed on a shaker (20 min, 4° C., ˜1000 rpm).

7. The plate was centrifuged at 4000×g for 5 min at 4° C. to precipitate debris.

8. 400 μl of the 500 μl volume was transferred to an auto-sampler plate.

9. ADC/IS calibrators and samples (in 100% MeOH) were evaporated under N₂ gas at 40° C. until the plate was dry.

10. The sample was reconstituted in 1000 μl of 95/5 CH₃CN(acetonitrile, CAN)/H₂O in 0.1% formic acid (FA) or 20% acetonitrile in 0.1% FA. The step was dependent on the type of chromatography used.

Sample Analysis
1. The LC column and mass spectrometer were equilibrated.
2. 20 μL of reconstituted sample was injected into the LC.
3. An LC column was used that provides appropriate chromatography for the released analytic target, coupled directly to a mass spectrometer, the analytic target and internal standard fragment ions were monitored using the multiple reaction monitoring (MRM) operation method.
4. The peak area for the analytic target was divided by the peak area obtained for the internal standard analytic target. The resultant analytic target/IS peak area ratio was plotted as a function of analytic target calibrator concentration (ng/ml), and points were fit to a curve using linear regression. The response ratios measured from the samples were quantified using the equation of the line determined by the standard curve.

The following analysis was directed specifically to release of MMAE, as the analytic target, from an Antibody conjugated to DPR-PEG-gluc-carbamate-MMAE.
1. The liquid chromatograph was equipped with a 50×3.0 mm 5 μm Silica column (BETASIL™, ThermoFisher Scientific) coupled to a tandem mass spectrometer, both of which were equilibrated.
2. 20 μL of reconstituted sample was injected
3. The following gradient of mobile phase A (0.1% formic acid in H₂O) and mobile phase B (0.1% formic acid in ACN) (Table 1) was used, with expected MMAE (analytic target) retention time at 1.16 minutes.

TABLE 1
LC Gradient

| Time (mins) | Flow (mL/min) | % A | % B |
|---|---|---|---|
| 0.1 | 1.0 | 5 | 95 |
| 0.25 | 1.0 | 5 | 95 |
| 1.00 | 1.0 | 80 | 20 |
| 1.70 | 1.0 | 80 | 20 |
| 1.80 | 1.0 | 5 | 95 |
| 4.00 | 1.0 | 5 | 95 |

4. MMAE concentration was determined using a multiple reaction monitoring (MRM) LC-MS/MS assay that selectively monitors for the transitions of 718 m/z to 686 m/z (precursor and fragment ion of MMAE) and 726 m/z to 694 m/z (precursor and fragment ion of d8-MMAE).
5. The peak area for each MMAE standard was divided by the peak area obtained for the internal standard d8-MMAE. The resultant MMAE/d8-MMAE peak area ratio was plotted as a function of MMAE standard concentration (ng/ml), and the points were fitted to a curve using linear regression. The response ratios measured from the samples were quantified using the equation of the line determined by the standard curve. For MMAE measurements, LC-MS/MS data were acquired and processed using operating and data analysis software available from the LC-MS/MS instrument manufacturer (Analyst® 1.6.1 and Multiquant version 2.1, AB SCIEX).

### Example 2: Ex Vivo Stability of Antibody-Drug Conjugates

The ex vivo stability of two ADCs was evaluated using the acid-catalyzed hydrolysis method. ADC1 and ADC2 are antibodies that are conjugated to mcMIVIAF, a potent anti-mitotic and anti-tubulin auristatin derivative (monomethyl auristatin F) that employs a maleimidocaproyl linker (mc).

A maleimide linker to a drug is generally represented as:

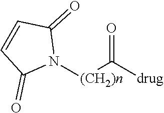

The mc linker is the above linker where n is 5. mcMIVIAF is the above species where n is 5 and drug is MMAF.

The mcMMAF linker-drug combination is not enzymatically cleavable.

STOCKS:

ADC1—7.1 mg/mL in PBS (4.3 drug/mAb; 203.5 uM mcMMAF equiv.)
IS1—4.4 mg/mL in PBS (4.0 drug/mAb; 117.3 uM labeled mcMIVIAF equiv.)
ADC2—5.5 mg/mL in PBS (4.1 drug/mAb; 150.3 uM mcMMAF equiv.)
IS2—at 9.0 mg/mL in PBS (3.8 drug/mAb; 228.0 uM labeled mcMIVIAF equiv.)

4 ml of Na⁺ citrated Sprague-Dawley rat plasma was spiked with ADC1 or ADC2 at a concentration of 250 μg/ml. 1 ml of the spiked plasma was used for generating a standard curve. The standard curve samples included serial dilutions of either ADC1 or ADC2.

From the remaining 3 ml of spiked plasma, 450 μl was removed at time points 0 hour, 6 hours, 1 day, 2 days, 4 days, and 7 days. Internal standards for ADC1 (IS1) and ADC2 (IS2) were prepared. The internal standards each included a ¹³C-labeled drug (specifically the phenyl ring of MMAF is labeled with 6 ⁿC), adding 6 amu to the mass. The internal standards were diluted to 10 μM drug-linker equivalents ([5×] concentration) into citrated rat plasma.

Preparation of 96-Well Sample/Standard Pre-Plate.

200 μl of each ADC sample and each standard curve sample were mixed 3 times and pre-plated into a 96-well, 350 μI/well plate by reverse pipetting. 50 μl of the internal standards were added to the ADC samples and standard curve samples. Each plated sample was mixed 3-5 times.

Preparation of 96-Well Filter Plate.

Protein A agarose was washed and equilibrated in PBS, pH 7.4 at a slurry ratio of 1 part resin to 3 parts buffer (200 μl resin bed in 800 μl slurry). 800 μl slurry volume of Protein A agarose was added to the appropriate locations on a 96-well filter plate. The plate was centrifuged at 1250×g for 5 minutes at 4° C. to remove the aqueous phase.

200 μl of each ADC sample and standard curve sample were mixed 3 times, then transferred to the appropriate location on the filter plate by reverse pipetting.

The plate was secured to a titer plate shaker set at 750-1000 rpm for 1 hour at 4° C.

Flow through fractions from the 96-well filter plate were recovered into a 96-well, 2 ml collection plate by centrifugation at 2000×g for 5 minutes at 4° C.

Each resin bed was washed once with 200 μl of wash buffer (40 mM KPO₄, 20 mM EDTA). The wash fractions were recovered by centrifugation at 2000×g for 5 minutes, 4° C. and set aside.

ADC Elution.

200 μl of IgG elution buffer was added to each resin bed, and the plate was placed on a Thermomixer at room temperature for 5 minutes at ˜1000 rpm. The eluant was recovered into a 2 ml collection plate by centrifugation at 2000×g for 5 minutes at 4° C. Another 200 μl of elution buffer was added to each resin bed, and the plate was placed on a Thermomixer at room temperature for 5 minutes at ˜1000 rpm. The eluant was recovered into a 2 ml collection plate by centrifugation at 2000×g for 5 minutes at 4° C. This yielded a final elution volume of 400 μl.

The eluant was evaporated under N₂ gas at 60° C. for 3-4 hours.

After evaporation, 400 μl of 10% v/v trifluoroacetic acid (TFA) (diluted in water) was added to each well. A Teflon-coated silicone plate mat was used to seal the 96-well plate. The plate was placed into a jacketed Thermomixer and incubated overnight (˜16 h) at 70° C. at ˜850 rpm.

The 96-well plate was subjected to a hard-spin (4000×g, 5 minutes) to pellet the protein precipitate. 300 μl was recovered and transferred to a new 96-well, 2 ml collection plate. The plate was evaporated under N₂ gas at 40° C. for 2-3 hours.

Each sample was resuspended in 300 μl of 33% CH₃CN (acetonitrile)/0.1% v/v formic acid to dissolve, and vortexed at ˜1000 rpm for 3 minutes.

The plate was spun for 5 minutes at 500×g, and 200 μl of each sample was transferred to an HPLC vial with silanized glass insert.

25 μl of each sample was analyzed via a quadrupole-time of flight (Q-TOF) mass spectrometer.

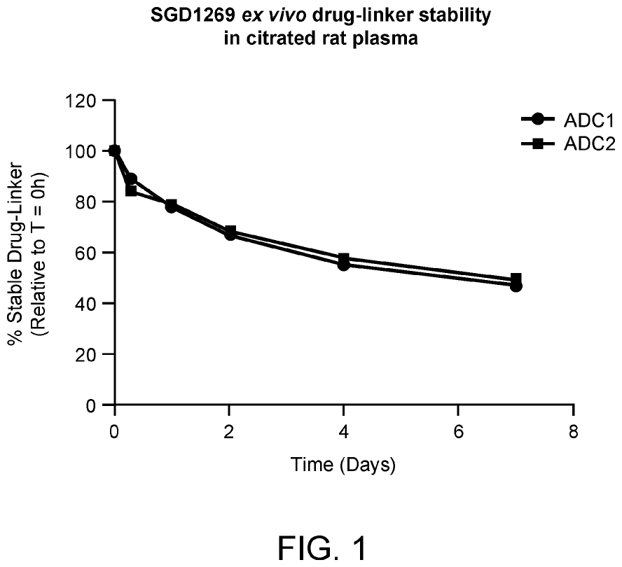

All time points and the corresponding standard curves were processed, and the concentration of released MMAF was determined. The results are shown in FIG. 1.

### Example 3: Clinical Sample Analysis

Clinical samples from patients treated with ADC3 were analyzed. ADC3 is an mcMMAF-conjugated antibody.

STOCKS:

ADC3—15 mg/mL in PBS 4 drugs/mAb
IS3—4.59 mg/mL in PBS (3.6 drug/mAb; 110.2 μM mcMMAF equiv.)

ADC3 standard curve samples were diluted into K2EDTA human plasma. The internal standard (diluted to 50 μM ADC equivalents) was also diluted into K2 EDTA human plasma, allowing for 100 μl per sample/standard.

Preparation of 96-Well Sample/Standard Pre Plate.

100 μl of each sample and standard curve sample were mixed and pre-plated into a 96-well. 200 μl of the internal standards was then added to each sample/standard curve sample. 500 μl of PBS-T was added to each well.

Preparation of 96-Well Filter Plate.

Protein A agarose was washed and equilibrated in PBS, pH 7.4 at a slurry ratio of 1 part resin to 3 parts buffer (200 μl resin bed in 800 μl slurry) and stored as a stock solution at 4° C. prior to use. 800 μl slurry volume of agarose (200 μl resin bed) was added to the appropriate locations on a 96-well filter plate. The plate was centrifuged at 1250×g for 5 minutes at 4° C. to remove the aqueous phase.

700 μl of each sample/standard curve sample/PBS-T were mixed 3 times, then transferred to the appropriate location on the filter plate by reverse pipetting.

The plate was secured to a titer plate shaker set at 750-1000 rpm 1 hour at 4° C.

Flow through fractions from the 96-well filter plate were recovered into a 96-well, 2 ml collection plate by centrifugation at 2000×g for 5 minutes at 4° C.

Each resin bed was washed once with 200 μl of PBS. The wash fractions were recovered by centrifugation at 2000×g for 5 minutes, 4° C. and set aside.

ADC Elution.

200 μl of IgG elution buffer was added to each resin bed, and the plate was placed on a Thermomixer at 4° C. for 5 minutes at 1000 rpm.

The eluant was recovered into a 2 ml collection plate by centrifugation at 2000×g for 5 minutes at 4° C. Another 200 μl of IgG elution buffer was added to each resin bed, and the plate was placed on a Thermomixer at 4° C. for 5 minutes at 1000 rpm. The eluant was recovered into the 2 mL collection plate by centrifugation at 2000×g for 5 minutes at 4° C. This yielded a final elution volume of 400 μL.

40 μl of 100% TFA was added to each well giving 10% v/v TFA to release the tetrapeptide analytic target. A Teflon™-coated silicone plate mat was used to seal the 96-well plate. The plate was placed into a jacketed Thermomixer and incubated overnight at 70° C. at ˜850 rpm in a chemical fume hood.

The 96-well plate was subjected to a hard-spin (4000×g, 5 minutes) to pellet the protein precipitate. 300 μl was recovered and transferred to a new 96-well, 2 ml collection plate. The plate was evaporated under N₂ gas at 40° C. for 2-3 hours.

Each sample was resuspended in 100 μl of 2% acetonitrile+0.1% formic acid to dissolve, and vortexed at 1000 rpm for 3 minutes.

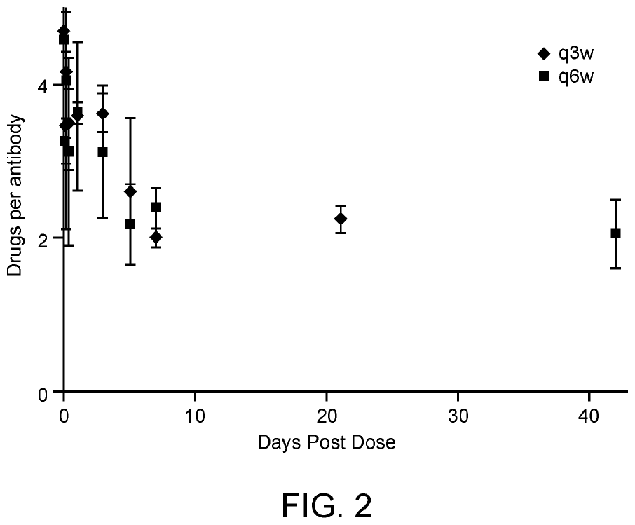

The analytic targets in the samples were analyzed using LC-MS/MS. The amount of antibody in the samples was measured using an ELISA assay. The results are shown in FIG. 2, plotted as drugs per antibody.

### Example 4: In Vivo Stability of Antibody-Drug Conjugate (Analyte Fragment)

The stability of ADC4 was analyzed in rats treated with ADC4 at 10 mg/kg or 20 mg/kg. ADC4 is a pegylated monomethyl auristatin E (DPR-PEG-gluc-carbamate-MMAE)-conjugated antibody. The MMAE linker is pegylated and contains diaminoproprionic acid and β-glucuronide which is cleavable by β-glucuronidase, see structure below. The acid release product is MMAE (analytic target).

The mal-peg-carbamate-MMAE has structure:

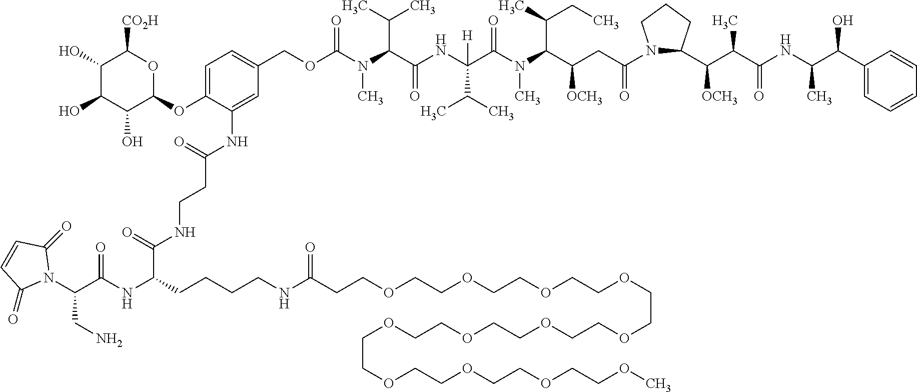 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCNC(=O)[C@H](CCCCNC(=O)CCOCCOCCOCCOCCOCCOCCOCCOCCOCCOCCOCCOC)NC(=O)[C@H](CN)N2C(=O)C=CC2=O)c1)C(C)C]

It is believed that the conjugation of the carbonate of the linker to the MMAE to form a carbamate facilitates cleavage of the entire MMAE drug on treatment with TFA.

STOCKS:

ADC4 at 5.4 mg/mL (36.0 uM ADC) in PBS (7.93 drug/mAb; 285.5 MMAE equiv.)
IS4 at 7.03 mg/mL (46.9 uM ADC) in PBS (7.93 drug/mAb; 371.9 uM d8-1\411\4AE equiv.)

K2EDTA rat plasma was spiked with ADC4 and internal standard. The internal standard included a ²H-labeled MMAE, adding 8 Da to the mass. MMAE standard curve samples were also prepared.

A pre-plate was prepared (see Example 1).

Preparation of 96-Well Filter Plate.

Protein A agarose was washed and equilibrated in PBS, pH 7.4 at a slurry ratio of 1 part resin to 3 parts buffer (200 μl resin bed in 800 μl slurry). 800 μl slurry volume of agarose (200 μl resin bed) was added to the appropriate locations on a 96-well filter plate. The plate was centrifuged at 1250×g for 5 minutes at 4° C. to remove the aqueous phase.

200 μl of each standard from the pre-plate were mixed 3 times, then transferred to the appropriate location on the filter plate.

The plate was secured to a titer plate shaker set at ˜900 rpm for 1 hour at 4° C.

Flow through fractions from the 96-well filter plate were recovered into a 96-well, 2 ml collection plate by centrifugation at 2000×g for 5 minutes at 4° C.

Each resin bed was washed two times with 400 μl of 1×PBS, pH 7.4. The wash fractions were recovered by centrifugation at 2000×g for 5 minutes, 4° C. and set aside.

ADC Elution. 200 μl of IgG elution buffer was added to each resin bed, and the plate was placed on a titer plate mixer at room temperature for 5 minutes at ˜900 rpm.

The eluant was recovered into a 2 ml collection plate by centrifugation at 2000×g for 5 minutes at 4° C. Another 200 μl of IgG elution buffer was added to each resin bed, and the plate was placed on a titer plate mixer at room temperature for 5 minutes at ˜900 rpm. The eluant was recovered into the 2 mL collection plate by centrifugation at 2000×g for 5 minutes at 4° C. This yielded a final elution volume of 400 μL.

The samples were evaporated under N₂ gas at 60° C. for 3-4 hours.

After evaporation, 400 μl of 10% TFA (diluted in H₂O) was added to each well. A Teflon-coated silicone plate mat was used to seal the 96-well plate. The plate was placed into a jacketed Thermomixer and incubated overnight (˜16 hours) at 70° C. at ˜650 rpm.

The 96-well plate was centrifuged at 2000×g, 5 minutes to spin down condensation from the sides of the wells. The plate as evaporated N₂ gas at 40° C. for ˜4 hours.

After evaporation, 500 μl of ice cold MeOH was added to each well. The plate was covered with a plate sealer and placed on a titer plate shaker for 20 minutes at 4° C.

The plate was subjected to a hard spin (4000×g for 5 minutes). 400 μl (out of the 500 μl total volume) was transferred to an auto-sampler plate.

The auto-sampler plate was evaporated under N₂ gas at 40° C. until dry. The samples were reconstituted in 1000 μl of 95/5 acetonitrile/H₂O in 0.1% FA.

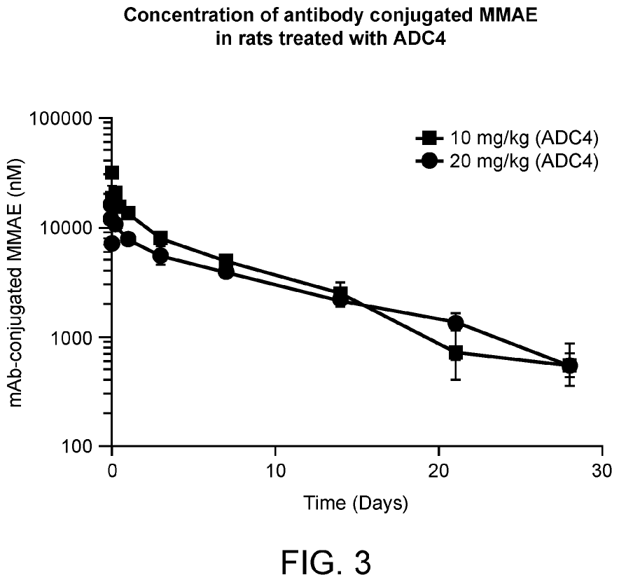

The samples were analyzed using LC-MS/MS. The results are shown in FIG. 3.

### Example 5: Development of Antibody-Drug Conjugate with Enhanced Stability

A collection of engineered cysteine antibodies (S239C, E269C, K326C and A327C) that can be site-specifically conjugated to potent cytotoxic agents was generated and their stability was tested by methods provided herein. In the experiment, homogenous 2-loaded ADCs with near 100% stability were identified. Furthermore, it was observed that stability of ADCs correlate with apparent hydrophobicity as measured by HIC, suggesting chemical sequestration as an additional means to confer stability without catalyzing thiosuccinimide hydrolysis.

Structural Analysis and Molecular Modeling

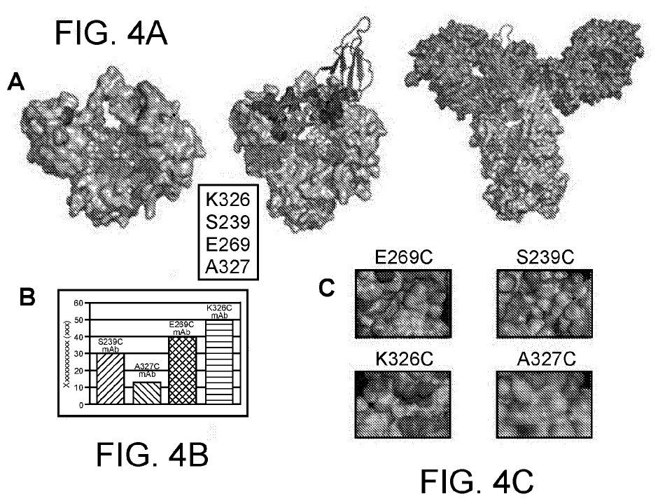

Protein database files of an intact antibody and of a human Fc region bound to Fc gamma receptor 3 (accession numbers 1HZH and 1E4K respectively) were used in analysis. Pymol (Schrodinger, 2010) was used to generate molecular structure model as provided in FIG. 4A. Using the human Fc region bound to Fc gamma receptor 3 (accession number 1HZH) as a template, selected residues (K326, 5239, E269 and A327) were converted to cysteine in silico and solvent accessibility for the new residue was calculated using GETAREA (Fraczkiewicz and Braun, 1998). The solvent accessibility for the four residues are presented in FIG. 4B, showing up to 5-fold difference between sites. Additionally, electrostatic calculations were carried out for the engineered cysteine antibodies (S239C, E269C, K326C and A327C) using APBS (Baker et al., 2001) and presented in FIG. 4C.

Conjugate Preparation

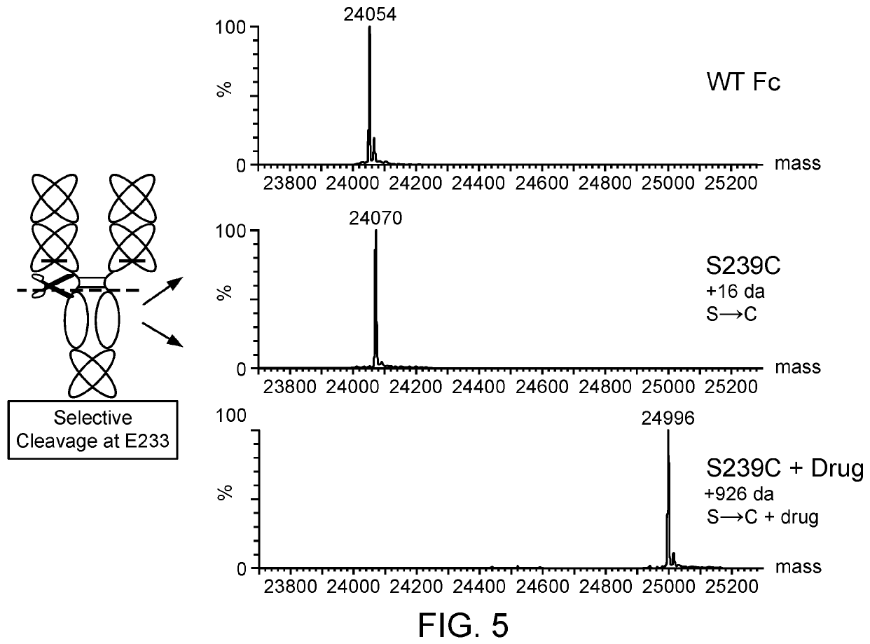

Humanized anti-CD70 (h1F6) (McEarchern et al., 2008) engineered with additional heavy chain cysteine residues (S239C, E269C, K326C and A327C) were conjugated to non-cleavable auristatin, maleimidocaproyl monomethylauristatin F (mcMMAF), and protease-cleavable pyrrolobenzodiazepine, or sandramycin (Biomar) drug linkers following protocols described previously (Jeffrey et al., 2013). Briefly, antibodies were fully reduced by adding 10 equivalents of tris(2-carboxyethyl)phosphine (TCEP) and 1 mM EDTA and adjusting the pH to 7.4 with 1M Tris buffer (pH 9.0). Following 1 hr incubation at 37° C., the reaction was cooled to 22° C. and 30 equivalents of dehydroascorbic acid were added. The pH was adjusted to pH 6.5 with 1 M Tris-HCl buffer (pH 3.7) and the oxidation reaction was allowed to proceed for 1 hr at 22° C. This resulted in reformation of native disulfides, but left the engineered heavy chain cysteines in the reduced state and available for conjugation. The pH of the solution was then raised again to pH 7.4 by addition of 1 M Tris buffer (pH 9.0). Conjugation was then carried out by the addition of 2.5 equivalents of the drug-linker, and the reaction was allowed to proceed at 22° C. for 30 min. The resulting conjugate was purified by gel filtration chromatography using a disposable PD-10 column (GE Healthcare). The degree of drug loading and site of drug attachment was determined by reducing the ADC with dithiothreitol followed by HPLC analysis on a PLRP column and integration of the heavy and light chain components (Sun et al., 2005). The extent of aggregation was determined by size exclusion chromatography. Analysis of intact ADCs using HPLC and mass spectrometry and methods as described in this disclosure confirmed a uniform population of ADCs with ˜2 drugs per mAb as expected (FIG. 5); in contrast wild-type mAbs do not incorporate any drug-linker under these conditions.

EC Site Conjugation Confirmation

Wild-type (WT Fc), engineered cysteine antibodies (S239C) and conjugated ADCs (S239C+Drug) were subjected to protease treatment with endoproteinase Glu-C (Sigma-Aldrich). Digestion with Glu-C resulted in liberation of the Fc fragment cleaved C-terminal of the hinge cysteines at position 233. Mass spectrometric analysis of this Fc fragment showed, when a wild-type ADC is digested, the resulting Fc fragment has a mass of 24,054 Da (FIG. 5, top panel) showing no signs of conjugation, consistent with all of the conjugation sites being on the N-terminal side of position 233. Digestion of an S239C antibody results in an Fc fragment with an additional 16 Da in mass, 24,070 Da total, corresponding to the difference in mass between serine and cysteine (FIG. 5, center panel). The digestion of a S239C pure 2-loaded ADC results in an Fc fragment with an additional 942 Da in mass, 24,995 Da total, corresponding to the differing masses of serine and cysteine and the addition of the drug linker (FIG. 5, bottom panel). The mass spectra (FIG. 5) demonstrate that only the mutant Fc regions incorporate a drug linker and that the introduced cysteine (S239C) is a novel site of conjugation. Similar results were seen with all of the engineered cysteine antibodies (E269C, K326C and A327C) and mass spectral analysis of the corresponding Fabs showed that all endogenous cysteines were present as disulfide bonds and had not been conjugated to the drug linker (data not shown).

Ex Vivo Maleimide Stability

IgG was removed from rat plasma (Bioreclamation IVT) by incubation with Protein A resin (ProSepA, Millipore), rotation overnight at 4° C., followed by filtration to remove the resin (Ultrafree-MC Centrifugal Filter, Millipore). ADCs were spiked into IgG-depleted plasma (0.25 mg/mL). Aliquots (200 μL) were removed immediately (t=0 d) and the remaining samples were incubated at 37° C. for up to 7 days. At relevant time points, test article and an internal standard were extracted and digested. A tetrapeptide product corresponding to N-terminal amino acids from MMAF (Val-Dil-Dap-Phe) was purified using solid phase extraction and quantified with reference to a standard by quadrupole-time-of-flight (QTOF) mass spectrometry. Methods of this disclosure were employed to release the tetrapeptide from the ADC and quantify the amount released.

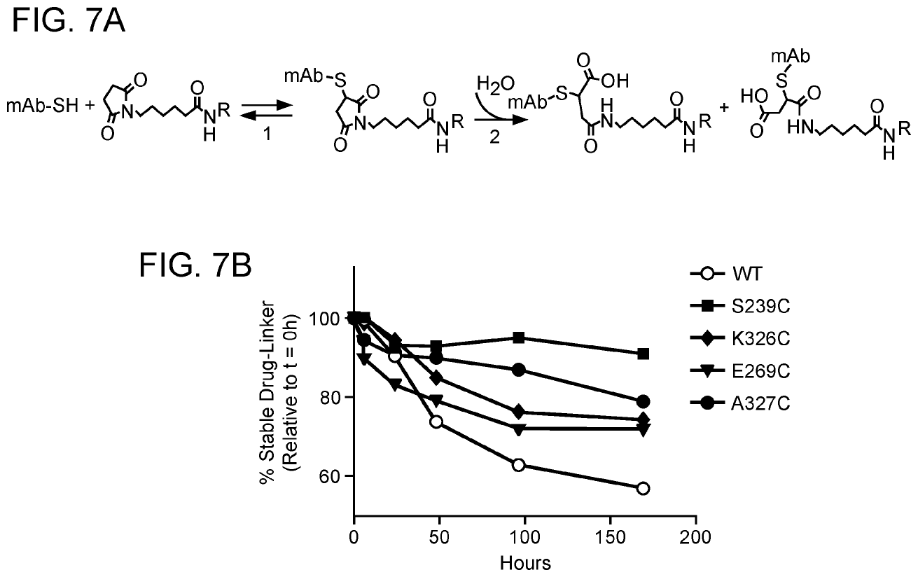

We found that the propensity of the maleimide to undergo retro-Michael loss of the drug conjugate (FIG. 7A) depended on the site of conjugation, with the wild-type 4-drug loaded ADC being the most susceptible and losing roughly 40% of its drug load over the seven days. In comparison, S239C was the most stable, losing less than 10% over the same period. A327C, E269C and K326C showed intermediate levels of drug loss with 21%, 28% and 26% respectively (FIG. 7B).

Ex Vivo Maleimide Hydrolysis

IgG was removed from rat plasma (Bioreclamation IVT) by incubation with protein A resin (ProSepA, Millipore), rotating overnight at 4° C., followed by filtration to remove the resin (Ultrafree-MC Centrifugal Filter, Millipore). ADCs were spiked into IgG-depleted plasma (0.25 mg/mL). Aliquots (500 μL) were removed immediately (t=0 d) and the remaining samples were incubated at 37° C. for 7 days. Each sample (500 μL) was rotated with 300 μL ProSepA resin to capture ADC (50% PBS slurry, 4° C. overnight) and then filtered through Ultrafree-MC spin cups (1 min, 11,000×g). The resin-bound ADC was washed with PBS (3×500 μL) and eluted with 300 μL IgG elution buffer (Thermo Scientific) into 30 μL 1 M Tris pH 8. A sample of each eluate (100 μL) was treated with 1 μL PNGase F (500 U/μL, New England Biolabs) and 5 μL LysC (0.1 μg/μL, Promega) at room temperature for 30 min, followed by 37° C. for 25 min, and subsequent addition of 100 mM dithiothreitol (10 μL) with further 15 min incubation at 37° C. The samples were examined using LC-MS via PLRP-S chromatography and electrospray ionization QTOF mass spectrometry. Data was deconvoluted using the MaxEnt1 function in MassLynx 4.0. Peak heights of deglycosylated HC Fc plus mcMMAF and deglycosylated HC Fc plus mcMMAF plus water were used for calculation of percent hydrolyzed drug linker.

Pre-incubation samples showed a consistent amount of maleimide hydrolysis (˜15%), indicated by an increase in mass of ˜20 Da, for all mutant ADCs. Post-incubation samples had dramatically different levels of additional ring opening: 7%, 9%, 61% and 65% respectively for S239C, A327C, E269C and K327C (Table 3). This result represents a nearly perfect inverse correlation between stability against retro-Michael elimination and hydrolytic rate enhancement by the drug linker's chemical microenvironment.

TABLE 3
Drug linker stability, maleimide hydrolysis,
and hydrophobicity of ADCs

| | | % Drug | % Maleimide | HIC Retention |
|---|---|---|---|---|
| | Load | Retained | Hydrolysis | Time |
| Wild-type mAb | 0 | N/A | N/A | 17 |
| Wild-type ADC | 4 | 57 | N/A | 25.2 |
| S239C | 2 | 91 | 7 | 17.5 |
| A327C | 2 | 79 | 9 | 20.4 |
| E269C | 2 | 72 | 61 | 22.3 |
| K326C | 2 | 74 | 65 | 23.4 |

Biophysical Characterization of Conjugation Sites

In order to investigate the origin of these differential rates of hydrolysis and stabilization of the thiosuccinimide bond, we looked at several different attributes of the engineered conjugation sites: 1) the electrostatic environment surrounding the conjugation sites (FIG. 4C); 2) calculated solvent accessibility of engineered cysteine (FIG. 4B); and 3) apparent hydrophobicity of each of the engineered ADCs (Table 3).

Local charged residues might promote or prevent proton abstraction and result in stabilization or elimination of the maleimide conjugate. We therefore analyzed the solvent accessible surface of the engineered Fc domains models by electrostatic potential. Visual inspection of these potentials surrounding the conjugation site showed no consistent placement of ionizable residues that could promote ring opening in the K326C and E269C ADCs. In fact, these introduced cysteines are in basic and acidic environments, respectively, and exhibited no difference in stability or susceptibility to hydrolysis. Nor did we find charged residues that could stabilize S239C and A327C, which are also in basic and acidic environments, respectively.

Conjugate hydrolysis requires accessibility of solvent molecules to the carbonyl atoms of the thiosuccinimide. It is possible that the maleimide conjugated at position 239 is simply shielded from the solvent and cannot participate in such a reaction. To determine whether solvent accessibility to conjugation sites predicted propensity of thiosuccinimide hydrolysis, we calculated the Connolly surface of in silico generated models. We found no correlation between exposed surface area and rates of hydrolysis (FIG. 4B). However, when using hydrophobic interaction chromatography (HIC), a correlation was found between retention time of the conjugates and drug-linker stability. This assay shows that the engineered ADCs that are least stable and quickest to hydrolyze (E269C and K326C) also exhibit the greatest apparent hydrophobicity.

Competition Binding

1×10⁵ antigen expressing cells (786-0) in PBS were aliquoted in each well of 96-well V-bottom plates on ice. The cells were incubated for 1 hr with 600 nM AlexaFluor-647 labeled wild-type m1F6 and increasing concentrations (from 0.19 nM to 600 nM) of unlabeled mutants or wild type ADCs. Cells were pelleted and washed 3 times with PBS. The cells were then pelleted and resuspended in 125 μL of PBS/BSA. Fluorescence was analyzed by flow cytometry, using percent of saturated fluorescent signal to determine percent labeled antibody bound and to subsequently extrapolate the EC₅₀ by fitting the data to a sigmoidal dose-response curve with variable slope.

The concentration at which the competitor antibody reduces the fluorescent signal by 50% is reported as the IC₅₀ in Table 2. The wild-type and cysteine mutants are identical within the error of the measurements indicating that cysteine substitutions and subsequent conjugation have no effect on engagement of the target by the ADC. In addition to measuring relative affinities of this collection of ADCs, we examined the cytotoxic effect that they have on antigen expressing cells, Table 2. Surprisingly all mutant ADCs had similar potency to the wild-type ADC when incubated with CD70 positive cells, despite the fact that nominal drug dose for the mutant ADCs was only half that of the wild-type ADC.

TABLE 2
Binding and in vitro activity of mutant and wild-type ADCs

| | Drug load | IC₅₀ (nM) | EC₅₀ (ng/mL) |
|---|---|---|---|
| Wild-type | 4 | 12 | 71 |
| S239C | 2 | 14 | 97 |
| E269C | 2 | 12 | 73 |
| K326C | 2 | 9 | 123 |
| A327C | 2 | 16 | 148 |

Table 2 Legend: To quantify relative binding affinities, a fixed concentration of fluorescently labeled parental antibody was titrated with increasing concentrations of non-labeled mutant or parental antibody and applied to antigen expressing cells. The concentration at which the competitor antibody reduces the fluorescent signal by 50% is reported as the IC₅₀. To assess in vitro activity, increasing concentrations of ADC were applied to antigen expressing cells. The concentration of ADC that gives half-maximal response is reported as the EC₅₀.

In Vitro Cytotoxicity Activity Assay

786-0 cells were obtained from the American Type Culture Collection and propagated in culture conditions recommended by the manufacturer. Cells were plated in 150 μL growth media per well into black-sided clear-bottom 96-well plates (Costar, Corning). 24 hr later, drug stocks were titrated as 5-fold serial dilutions producing 8-point dose curves and added at 50 μl per well in duplicate. Cells were then incubated for 96 hr at 37° C., 5% CO₂. Cytotoxicity was measure by incubating with 100 μL Cell Titer Glo (Promega) solution for 0.5 hours, and then luminescence was measured on an EnVision Xcite plate reader (Perkin Elmer). Data was processed with Excel (Microsoft) and GraphPad (Prism) to produce dose response curves and IC₅₀ values were generated and data collected.

In Vivo Activity Studies

To establish the 786-0 model, 5×10⁶ cells were implanted into the right flank of athymic nu/nu female donor mice (Harlan). When donor tumors were ˜500 mm³ [(L×W²)/2], mice were euthanized, tumors were aseptically excised, and ˜0.5×0.5 mm fragments were loaded into a sterilized 13-gauge trochar for implantation into anesthetized mice. When tumors reached ˜100 mm³, mice were randomly allocated to treatment groups and dosed via intraperitoneal injection at a single time point with 10 mg/kg ADC. Tumors were measured twice weekly, and volumes were calculated using the formula V=(L×W²)/2. Animals were euthanized when tumors reached 1,000 mm³. Tumor volume was calculated using the formula, (A×B²)/2, where A and B are the largest and second largest perpendicular tumor dimensions, respectively. Mean tumor volume and weight of mice were monitored and mice terminated when the tumor volume reached 1,000 mm³.

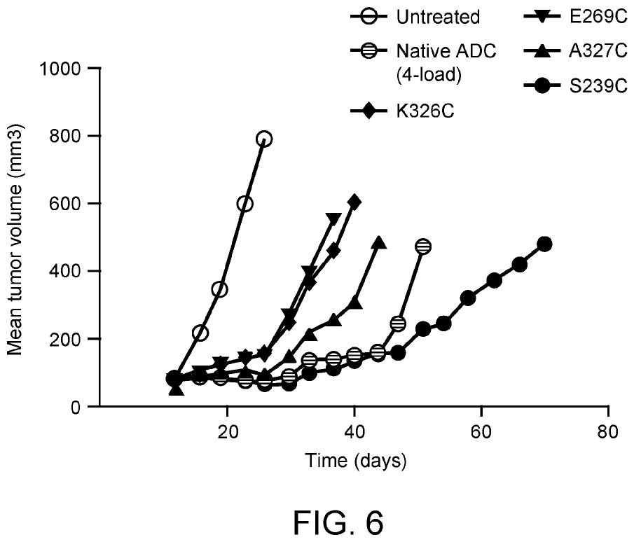

In a single dose 786-0 in vivo efficacy model (FIG. 6), all ADCs had a significant impact on growth of the tumor when compared to untreated mice. However, there was a difference in performance between the mutants. Out of the mutants, 2-loaded S239C showed the best tumor growth inhibition, delaying tumor growth for ˜25 days, similar to the wild-type 4-load ADC. While A327C was slightly less effective than S239C (delaying tumor growth for 10 days), it out-performed E269C and K326C which had identical activities and only delayed tumor growth for ˜5 days.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

The present disclosure provides, inter alia, compositions of cannabinoid and entourage compositions. The present disclosure also provides method of treating neurodegenerative diseases by administering the cannabinoid and entourage compositions. While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A method of analyzing a ligand-drug conjugate (LDC) in a sample, comprising the step of:
a. providing the sample comprising the LDC, wherein the LDC comprises a ligand and an analytic target, wherein the analytic target comprises a drug molecule or a portion thereof; and
b. contacting the sample with aqueous trifluoroacetic acid (TFA) at a concentration between 1 to 30% (v/v), thereby inducing release of the analytic target from the LDC.

2. The method of claim 1, further comprising the steps of:
a. measuring the amount of the analytic target released from the LDC; and
b. determining the concentration of the drug molecule or the portion thereof in the sample using the amount of the released analytic target.

3. The method of claim 2, wherein the step of measuring the amount of the analytic target released from the LDC comprises subjecting the analytic target to liquid chromatography-mass spectrometry (LC-MS).

4. The method of claim 2, wherein the step of measuring the amount of the analytic target released from the LDC comprises subjecting the analytic target to liquid chromatography tandem mass spectrometry (LC-MS/MS).

5. The method of any of claims 2-4, further comprising the steps of:
a. measuring the amount of the ligand in the sample; and
b. determining the concentration of the drug molecule or the portion thereof in the sample by using the measured amount of the ligand.

6. The method of any of claims 1-5, further comprising the step of collecting the LDC from the sample prior to the step of contacting the sample with aqueous trifluoroacetic acid (TFA).

7. The method of claim 6, wherein the step of collecting the LDC is performed by affinity chromatography, size exclusion chromatography, ammonium sulfate precipitation, ion exchange chromatography, immobilized metal chelate chromatography, or immunoprecipitation.

8. The method of any of claims 2-7, wherein the step of measuring the amount of the analytic target released from the LDC is performed by using a standard curve of the LDC.

9. The method of any of claims 1-8, further comprising the steps of:
a. adding to the sample a fixed amount of an internal standard, wherein the internal standard comprises the ligand and a second analytic target, wherein the second analytic target is a labeled derivative of the LDC;
b. contacting the sample with aqueous trifluoroacetic acid (TFA) at a concentration between 1 to 30% (v/v), thereby inducing release of the analytic target from the LDC and the second analytic target from the internal standard;
c. measuring the amount of the second analytic target released from the internal standard; and
d. measuring the amount of the analytic target released from the LDC based on the amount of the second analytic target released from the internal standard.

10. The method of claim 9, wherein the second analytic target has a different molecular weight than the analytic target.

11. The method of any of claims 9-10, wherein the internal standard comprises an isotopically labeled version of the LDC.

12. The method of claim 11, wherein the isotopic label is stable or non-stable.

13. The method of claim 12, wherein the isotopic label is deuterium or carbon 13.

14. The method of any of claims 9-13, further comprising the step of: collecting the LDC and the internal standard from the sample prior to the step of contacting the sample with aqueous trifluoroacetic acid (TFA).

15. The method of claim 14, wherein the step of collecting the LDC or the internal standard is performed by affinity chromatography, size exclusion chromatography, ammonium sulfate precipitation, ion exchange chromatography, immobilized metal chelate chromatography, or immunoprecipitation.

16. The method of claim 7 or 15, wherein the ligand is an antibody or a functional fragment thereof and the LDC or the internal standard are collected from the sample by contacting the sample with a resin selected from a Protein A resin, a Protein G resin and a Protein L resin.

17. The method of any of claims 1-16, wherein the sample is contacted with aqueous trifluoroacetic acid (TFA) at a concentration of 10% (v/v).

18. The method of any of claims 1-17, wherein the drug molecule is monomethyl auristatin E (MMAE) or monomethyl auristatin F (MMAF).

19. The method of claim 18, wherein the drug molecule is monomethyl auristatin F (MMAF).

20. The method of any of claims 1-19, wherein the analytic target comprises a tetra-peptide, Val-Dil-Dap-Phe.

21. A method of determining stability of the ligand-drug conjugate (LDC), comprising the steps of:
a. obtaining a first sample and a second sample from a single source at different time points after exposure to the LDC;
b. analyzing the LDC in the first sample and the second sample by the method of any of claims 2-20, thereby determining the amounts of the analytic target released form the LDC in the first sample and the second sample; and
c. determining stability of the LDC by comparing the amounts of the released analytic target in the first sample and the second sample.

22. The method of claim 21, further comprising the steps of:
a. measuring the amounts of the ligand in the first sample and the second sample; and
b. determining the ratios of the amount of the released analytic target and the ligand in the first sample and the second sample.

23. The method of any of claims 1-22, wherein the sample, the first sample, or the second sample is a biological sample derived from mammalian tissues or aqueous mammalian fluids.

24. The method of claim 23, wherein the biological sample is obtained from one of the following: plasma, serum, blood, tissue, tissue biopsy, feces, and urine.

25. The method of claim 24, wherein the biological sample is obtained from plasma.

26. The method of claim 25, wherein the plasma was treated with the LDC.

27. The method of any of claims 25-26, wherein the plasma is from a human subject that has been treated with the LDC.

28. A method for quantifying an LDC in a sample, comprising the steps of:
a. providing a sample comprising the LDC, wherein the LDC comprises an analytic target, the analytic target comprising a drug molecule;
b. adding to the sample an internal standard, wherein the internal standard is a labeled derivative of the LDC and comprises a second analytic target;
c. extracting the LDC and the internal standard from the sample;
d. contacting the LDC and the internal standard with aqueous TFA at a concentration between 1 to 30% (v/v), wherein the TFA releases the analytic target from the LDC and the second analytic target from the internal standard;
e. determining the amount of the analytic target released from the LDC and the second analytic target released from the internal standard, wherein the amount of the analytic target released from the LDC correlates with the amount of LDC in the sample.

29. The method of claim 28, wherein the amount of the analytic target released from the LDC is determined by using the amount of the second analytic target released from the internal standard, wherein the amount of analytic target released from the LDC correlates with the concentration of the drug molecule conjugated to an antibody in the LDC in the sample.

30. The method of any of claims 28-29, wherein the amount of the analytic target released from the LDC is determined by using a standard curve of the LDC.

31. The method of any of claims 28-30, wherein the drug molecule is monomethyl auristatin F (MMAF) or monomethyl auristatin E (MMAE).

32. The method of any of claims 28-31, wherein the analytic target comprises MMAF or tetra-peptide Val-Dil-Dap-Phe.

33. The method of any of claims 28-32, wherein the analytic target comprises mcMMAF.

34. The method of any of claims 28-32, wherein the analytic target and the second analytic target comprises tetra peptide Val-Dil-Dap-Phe and the second analytic target is isotopically labeled with 6 or more carbon and 13 or 6 or more deuterium.

35. The method of any of claims 28-32, wherein the analytic target and the second analytic target comprises a pegylated linker DPR-PEG-gluc-carbamate-MMAE.

36. The method of any of claims 28-32, wherein the analytic target and the second analytic target comprises MMAE and the second analytic target is isotopically labeled with 6 or more carbon and 13 or 6 or more deuterium.

37. The method of any of claims 28-36, wherein the LDC and the internal standard are contacted with the aqueous TFA concentration at a concentration of 10% v/v.

38. A kit for determining the amount of an LDC in a sample, comprising:
a. an internal standard for the LDC, wherein the internal standard is a labeled derivative of the LDC, and comprises a drug molecule; and
b. aqueous trifluoroacetic acid TFA for application at a selected concentration between 1 to 30% (v/v).

39. The kit of claim 38, wherein the internal standard is isotopically labeled.

40. A kit for determining the amount of an LDC in a sample, comprising:
a. a labeled linker-drug complex and a ligand, wherein the labeled linker-drug complex can be conjugated to the ligand, thereby forming an internal standard; and
b. aqueous trifluoroacetic acid TFA for application at a selected concentration between 1 to 30% (v/v).

41. The kit of claim 40, wherein the internal standard is isotopically labeled.
